(19) United States
(12) Patent Application Publication
(10) Pub. No.: US 2022/0160822 A1
(43) Pub. Date: May 26, 2022

(54) NEUTRALIZING VACCINES AGAINST HUMAN CORONAVIRUS

(71) Applicant: Wisconsin Alumni Research Foundation, Madison, WI (US)

(72) Inventors: Adel M. Talaat, Madison, WI (US); Shaswath Chandrasekar, Madison, WI (US)

(21) Appl. No.: 17/534,002

(22) Filed: Nov. 23, 2021

Related U.S. Application Data
(60) Provisional application No. 63/117,354, filed on Nov. 23, 2020.

(51) Int. Cl. A61K 38/16; A61P 31/14; C07K 14/165; C12N 7/00

(52) U.S. Cl. CPC A61K 38/162; A61P 31/14; A61K 2039/55583; C12N 7/00; C07K 14/165

(57) ABSTRACT

The present invention provides both QuilA-loaded chitosan (QAC)-encapsulated DNA vaccine compositions and viral vaccine compositions that encode a SARs-CoV-2 spike (S) protein, a SARs-CoV-2 nucleocapsid (N) protein, or both the S protein and the N protein. Additionally, the present invention provides methods in which the disclosed vaccines are administered to a subject to induce an immune response against SARS-CoV-2.

### CROSS-REFERENCE TO RELATED APPLICATIONS

This patent application claims the benefit of priority of U.S. Provisional Patent Application No. 63/117,354, filed Nov. 23, 2020, which is incorporated herein by reference in its entirety.

### STATEMENT REGARDING FEDERALLY SPONSORED RESEARCH

This invention was made with government support under project number 2019-05849 awarded by the United States Department of Agriculture. The government has certain rights in this invention.

### SEQUENCE LISTING

This application is being filed electronically via EFS-Web and includes an electronically submitted Sequence Listing in .txt format. The .txt file contains a sequence listing entitled “960296_04249_ST25.txt” created on Nov. 22, 2021 and is 38,375 bytes in size. The Sequence Listing contained in this .txt file is part of the specification and is hereby incorporated by reference herein in its entirety.

### BACKGROUND

COVID-19 represents a significant challenge to public health authorities worldwide because of the speed of disease transmission and the lack of effective treatment or prevention strategies (1). The development of traditional vaccines, such as inactivated or live-attenuated vaccines, is time-consuming and may not meet the need for rapid vaccine development. For the inactivated vaccines, there are safety concerns such as incomplete inactivation of the pathogen and the need to grow large volumes of deadly pathogens (2). On the other hand, live-attenuated vaccines have been shown to induce an antibody-dependent enhancement effect worsening the clinical outcome of rhesus macaques infected with SARS-CoV (3). Other vaccination strategies such as subunit, RNA-based, and viral vector vaccines have advantages over traditional vaccines. However, their use comes with many challenges. For example, subunit vaccines are expensive to produce due to limited global production capacity and typically have low immunogenicity (4).

Amidst a global pandemic, there is a need for rapid development of vaccines. Vectored vaccines (i.e., DNA and viral vaccines) are advantageous because plasmids encoding antigens can be developed within a few days using rapid and inexpensive gene synthesis technologies (5). For viral vectored vaccines, highly efficient, scaled-up production processes have already been established, enabling their use during a pandemic such as COVID-19 (6). Previously, experimental plasmid vaccines have been developed for emerging infections, including SARS-CoV, SARS-CoV-2, MERS coronavirus (CoV), Influenza A, and Zika virus (7). DNA based vaccines offer several advantages over other technologies discussed above, including rapid vaccine production that can be scaled up within a few days on demand (5). Further, multiple vaccine constructs can be used together (8) to immunize patients effectively, offering highly flexible vaccination programs. Importantly, synthetic DNA is temperature stable and does not need the cold chain needed for live attenuated or subunit vaccines, thus lowering the cost of vaccine administration and simplifying supply logistics in resource-limited countries.

The correlates of protection against COVID-19 are still ambiguous. Most vaccine efforts are focused on generating neutralizing antibodies, and little attention has been given to CD4+ and CD8+ T cell responses (13-16). Patients that have recovered from SARS infections had undetectable anti-SARS antibodies and memory B cells. However, their CD8+ T cells persisted many years after the infection (17, 18). A recent study showed that the antibody level in many SARS-CoV-2 recovered patients declined to baseline levels within three months, suggesting that the vaccines relying solely on a neutralizing antibody response may not confer long-term protection against SARS-CoV-2 and other coronaviruses (19). Thus, there remains a need in the art for vaccines against SARS-CoV-2 that can induce both humoral and cellular immune responses, as these vaccines should produce a more durable protective immune response.

### SUMMARY

Vaccines directed to the spike and nucleocapsid proteins of SARs-CoV-2 and methods of using the same are provided herein. A vaccine composition including an adjuvant and a polynucleotide that encodes a SARs-CoV-2 spike (S) protein or portion thereof, a SARs-CoV-2 nucleocapsid (N) protein or portion thereof, or both the S protein and the N protein is provided. In one aspect, the adjuvant comprises disaggregated spherical nanostructures comprising Quil-A and chitosan, and wherein the Quil-A and chitosan are present at a ratio between 1:15 and 1:100.

In another aspect, a vaccine composition including a viral vector comprising a polynucleotide encoding a SARs-CoV-2 spike (S) protein or portion thereof, a SARs-CoV-2 nucleocapsid (N) protein or portion thereof, or both the S protein and the N protein is provided. The viral vector may be an adenovirus, including an adeno-associated virus or a poxvirus such as a vaccinia virus.

In another aspect, methods of inducing an immune response against SARs-CoV-2 in a subject are provided. The methods include administering the vaccine compositions in an amount effective to induce the immune response against at least one SARS-CoV-2 antigen in the subject.

In a still further aspect, methods of inducing an immune response against a SARs-CoV-2 in a subject are provided. These methods may include administering a first vaccine composition comprising one of the vaccine compositions provided here to the subject and then after a period of time, administering a second vaccine composition comprising either the same or a different vaccine composition provided herein. The administration of the first vaccine composition and the second vaccine composition induces the immune response against at least one SARS-CoV-2 antigen in the subject. In one embodiment the first vaccine composition is a DNA vaccine and the second vaccine composition is a viral vector-based vaccine.

### BRIEF DESCRIPTION OF THE DRAWINGS

FIG. 1 shows maps of the SARS-CoV-2 vector vaccine constructs expressing S and N proteins. a) Gene map of pCMV plasmid expressing Truncated S (TrS) protein. b) Gene map of pCMV plasmid backbone expressing N protein. c) Gene map of the MVA vaccine construct expressing TrS protein and cytoplasmic tail (CT) with the addition of C-terminal 6×His tag. d) Gene map of the MVA construct expressing N protein. All gene maps were generated using Snapgene software. e) Western blot analysis with anti 6×His-HRP antibody (left) and polyclonal mouse anti-SARS-CoV-2 spike sera (right), confirming expression of S protein from vaccine constructs. Lanes are as follows: supernatant from HEK 293T cells transfected with control plasmid (lane 2), supernatant from HEK 293T cells transfected with pCMV-TrS plasmid (lanes 1 and 7), supernatant from CEF cells infected with MVA-TrS (lanes 3 and 6), control non-infected supernatant (lane 4), purified recombinant SARS-CoV-2 S glycoprotein (lane 5; BEI resources-NR-52396). f) Western blot analysis with anti 6×His-HRP antibody from N vaccine constructs. Lanes are as follows: cell pellet from HEK 293T cells transfected with control plasmid (lane 3), cell pellet from HEK 293T cells transfected with pCMV-N plasmid (lane 1), and cell pellet from CEF cells infected with MVA-N (lane 2).

FIG. 2 shows the generation of humoral immune responses in C57BL/6 mice following immunization with different vaccine constructs. a) Schematic depiction of the vaccine constructs and immunization protocol. Groups of C57BL/6 mice were vaccinated with 3 doses of pQAC-CoV (I.N) or pQAC-CoV (I.M) separated by 3 week intervals. Another group of C57BL/5 mice was vaccinated with pQAC-CoV (I.N) at week-0 followed by a boost with MVA-CoV (I.N) at week-6. b) ELISA titers of SARS-CoV-2 S-specific IgG in mice sera. c) ELISA titers of SARS-CoV-2 spike RBD-specific IgG in mice serum. d) ELISA titers of SARS-CoV-2 S-specific IgA in BAL. Significance (*, P<0.05, ****, P<0.0001) was determined by two-way ANOVA. Data show means±SEM.

FIG. 3 demonstrates that the heterologous vaccine strategy elicits robust binding antibody responses. Groups of C57BL/5 mice were either not vaccinated (PBS) or immunized with pQAC/MVA-CoV (I.N). Serum and BAL samples were collected 3 weeks post boost. a) SARS-CoV-2 spike-specific serum IgG binding endpoint titers measured by ELISA. b) Serial serum dilutions of IgG binding to SARS-CoV-2 spike protein. c) SARS-CoV-2 spike-specific BAL IgA binding endpoint titers measured by ELISA. d) Serial BAL dilutions of IgA binding to SARS-CoV-2 spike protein. Significance (*, P<0.05) was determined by one-way ANOVA. Data show means±SEM.

FIG. 4 demonstrates that the heterologous vaccine strategy elicits robust neutralizing antibody responses. Groups of C57BL/6 mice were either not vaccinated (PBS) or immunized with pQAC/MVA-CoV (I.N). Serum and BAL samples were collected 3 weeks post boost. a) 50% serum neutralization titer of pseudovirus expressing SARS-CoV-2 spike protein. b) Serum neutralization titer of wild-type SARS-CoV-2, Isolate USA-WA1/2020. c) 50% BAL neutralization titer of pseudovirus expressing SARS-CoV-2 spike protein. d) BAL neutralization titer of wild-type SARS-CoV-2, Isolate USA-WA1/2020. Significance (*, P<0.05; **, P<0.01) was determined by one-way ANOVA. Data show means±SEM.

FIG. 5 shows SARS-CoV-2 spike-specific T-cell responses in the spleen of vaccinated C57BL/6 mice. Intracellular cytokine staining was performed on spleens harvested 3 weeks after final boost to assess T-cell responses. a) Type 1 helper (Th1) responses (IFN-γ, TNFα, or IL-2+), b) Type 2 helper (Th2) responses (IL-13+), c) Type 17 helper (Th17) responses (IL-17+), (d) Type 1 cytotoxic (Tc1) responses (IFN-γ or TNFα or IL-2+), (e) Type 2 cytotoxic (Tc2) responses (IL-13+), and (f) Type 17 cytotoxic (Tc17) responses (IL-17+) were detected using intracellular cytokine staining assays for spleen T-cells in response to recombinant SARS-CoV-2 spike stimulation. Significance (*, P<0.05; **, P<0.01) as compared to PBS controls was determined by ANOVA. Data show means±SEM.

FIG. 6 shows SARS-CoV-2 spike-specific T-cell responses in the lungs of vaccinated C57BL/6 mice. Intracellular cytokine staining was performed on lungs harvested 3 weeks after final boost to assess T-cell responses. a) Type 1 helper (Th1) responses (IFN-γ, TNFα, or IL-2+), b) Type 2 helper (Th2) responses (IL-13+), c) Type 17 helper (Th17) responses (IL-17+), d) Type 1 cytotoxic (Tc1) responses (IFN-γ, TNFα, or IL-2+), e) Type 2 cytotoxic (Tc2) responses (IL-13+), and f) Type 17 cytotoxic (Tc17) responses (IL-17+) were detected using intracellular cytokine staining assays for lung T-cells in response to recombinant SARS-CoV-2 spike stimulation. Samples with lower numbers of live cells (<10,000) were excluded from analysis. Significance (*, P<0.05; **, P<0.01) as compared to PBS controls was determined by ANOVA. Data show means±SEM.

FIG. 7 demonstrates that immunization with SARS-CoV-2 vaccine constructs induces polyfunctional T-cells. a) CD4+ IFN-γ, TNFα, IL-2, and IL-17, b) CD 4+IL-2 and IL-17, c) CD8+IFN-γ, TNFα, IL-2, and IL-17 polyfunctional T-cells detected in the spleen of vaccinated mice. d) CD4+IFN-γ, TNFα, IL-2, and IL-17, e) CD8+IFN-γ and TNFα, f) CD8+IFN-γ, TNFα, and IL-17 polyfunctional T-cells detected in the lungs of vaccinated mice following intracellular cytokine staining assays after recombinant SARS-CoV-2 spike stimulation. Significance (*, P<0.05; **, P<0.01) as compared to PBS controls was determined by ANOVA. Data show means±SEM.

FIG. 8 shows a representative gating strategy for flow cytometry data. All samples (spleen and lung) had the following common gating: a) single cells, b) lymphocyte gate, c) live cells, d) CD4+ and CD8+ T-cells, e) IL17+ cells, f) IL-13+ cells, g) TNFα+ cells, h) IL-2+ cells and i) IFNγ+ cells.

FIG. 9 shows the SARS-CoV-2 Spike specific type-I responses in Spleens of vaccinated C57BL/6 mice. Intracellular cytokine staining was performed on spleens harvested 3 weeks after final boost to assess T-cell responses. (a) IL-2+, (b) IFN-γ, (c) TNFα+CD4+ T-cells and (d) IL-2+, (e) IFN-γ, (f) TNFα+CD8+ spleen T-cells in response to recombinant SARS-CoV-2 Spike stimulation.

FIG. 10 shows the SARS-CoV-2 Spike specific type-I responses in Lungs of vaccinated C57BL/6 mice. Intracellular cytokine staining was performed on lungs harvested 3 weeks after final boost to assess T-cell responses. (a) IL-2+, (b) IFN-γ, (c) TNFα+CD4+ T-cells and (d) IL-2+, (e) IFN-γ, (f) TNFα+CD8+ lung T-cells in response to recombinant SARS-CoV-2 Spike stimulation.

FIG. 11 Nanostructure of QAC encapsulated plasmid DNA. Number-based DLS data (a) and Zeta potential (b) on QAC-SARS-CoV-2 S nanoparticles at 25° C. with Zetasizer software. (c) Nanoparticles of QAC-SARS-CoV-2 S preparation (arrows) were seen with TEM. Scale bar, 50 nm (left) and 100 nm (right).

FIG. 12. QAC adjuvant system safety, release and delivery of DNA. (a) Viability of HEK 293T cells 72 hrs post addition of increasing amounts of pQAC-Luc as measured using MTT assay. (b) Expression of luciferase 72 hrs post addition of increasing amounts of pQAC-Luc. (c) Sustained release kinetics of packaged DNA in vitro measured at pH-7.4, 37° C. (d) Expression of Luciferase from released pCAG-Luc used in the release kinetics assay in comparison to fresh pCAG-Luc.

FIG. 13. Efficient internalization of QAC nanoparticles by J774 cells. Cell monolayers were incubated with Cy3 labeled (A) unencapsulated or (B) QAC encapsulated particles (green) for 4 or 24 hrs and stained for actin (Alexa phalloidin 546, red). DAPI (blue) was used to stain the nucleus. Representative images were captured by LSCM. Scale bars=5 μm.

FIG. 14. Generation of humoral immune responses in K18-hACE2 mice following immunization with different vaccine constructs. (a) Outline for vaccine construct and immunization protocol using groups of K18-hACE2 mice vaccinated with 2 doses of pQAC-CoV (IN) or pQAC-CoV (IM) with 6-week interval. Another group of K18-hACE2 mice were vaccinated with pQAC-CoV (IN) at week-0 followed by boost with MVA-CoV (IN) at week-6. (b) Serum neutralization (NAb) titer of wild-type SARS-CoV-2, isolate USA-WA1/2020, (c) Bronchoalveolar lavage (BAL) neutralization titer of wild-type SARS-CoV-2, isolate USA-WA1/2020 and (d) Serum NAb titer of wild-type SARS-CoV-2, isolate USA-WA1/2020 in comparison to UK (B.1.1.7) and SA (B.1.351) variants. Significance (*, p<0.05, **, p<0.01) was determined by two-way ANOVA. Data show mean±SEM.

FIG. 15. SARS-CoV-2 Spike specific T-cell responses in lungs of vaccinated K18-hACE2 mice. Intracellular cytokine staining was performed on lungs harvested 3 weeks after final boost to assess T-cell responses. (a) Type 1 helper (Th1) responses (IFN-γ or TNFα or IL-2+), (b) type 2 helper (Th2) responses (IL-13+), (c) type 17 helper (Th17) responses (IL-17+), (d) type 1 cytotoxic (Tc1) responses (IFN-γ or TNFα or IL-2+), (e) type 2 cytotoxic (Tc2) responses (IL-13+), (f) type 17 cytotoxic (Tc17) responses (IL-17+) intracellular cytokine staining assays for lung T-cells in response to recombinant SARS-CoV-2 spike stimulation. Significance (**, p<0.01) was determined by ANOVA compared to PBS controls. Data show mean±SEM.

FIG. 16. Protective efficacy of QAC based SARS-CoV-2 vaccines in K18-hACE2 mice. Three weeks following final immunization, K18-hACE2 mice were intranasally infected with 1×104 PFU of SARS-CoV-2. (a and c) Weight loss and (b and d) survival outcomes in K18-hACE2 transgenic mice. Data from follow-up trial depicted in c and d. Weight loss data show median with error (95% CI).

FIG. 17. Parenteral pQAC-CoV administration reduces lung tissue titer. SARS-CoV-2 titers in the lungs of vaccinated mice at 4 (a and c) and 6 (b and d) days post infection (dpi). Data from follow-up trial depicted in c and d. Significance (**, p<0.01; ***, p<0.001; ****, p<0.0001) or non-significance (ns) was determined by ANOVA compared to PBS controls (a and b) or student's t test (c and d). Data show mean±SEM.

FIG. 18. Parenteral pQAC-CoV administration prevents viral spread to the brain. SARS-CoV-2 titers in the brains of vaccinated mice at 4 (a and c) and 6 (b and d) days post infection (dpi). Viral titers measured using SARS-CoV-2 specific qRT-PCR (a and b) or infectious assay using VERO E6 cells (c and d). Data from follow-up trial depicted in c and d. Significance (**, p<0.01; ***, p<0.001; ****, p<0.0001) or nonsignificance (ns) was determined by ANOVA compared to PBS controls (a and b) or student's t test (c and d). Data show mean±SEM.

FIG. 19. Histopathologic analysis of SARS-CoV-2 infection in K18-hACE2 transgenic mice immunized with QAC based vaccines. Histology of fixed lung tissues, 6 days after SARS-CoV-2 infection. H&E-stained tissues (n=5 per group). Representative images of SARS-CoV-2-infected mice that received (a) PBS, (b) pQAC-CoV (IN), (c) pQAC-CoV (IM) or (d) pQAC/MVA-CoV (IN). Interstitial lung disease was reduced in the pQAC-CoV (IM). Scale bar, 252 μm. (e-f) Histopathologic scoring of lung tissues. Tissues from all four groups were ordinally scored for perivascular infiltrates, and interstitial lung disease. Error bars represent the SEM. *P<0.05 and **P<0.01, one-way ANOVA.

### DETAILED DESCRIPTION

The present invention provides both DNA vaccine compositions and viral vaccine compositions encoding a SARs-CoV-2 spike (S) protein, a SARs-CoV-2 nucleocapsid (N) protein, or both the S protein and the N protein or portions of at least one of these proteins. Further, the present invention provides methods in which the disclosed vaccines are administered to a subject to induce an immune response against SARS-CoV-2.

In the Examples, the inventors demonstrate that a novel heterologous vaccination strategy elicits robust immunity against SARS-CoV-2, inducing both humoral and cellular immune responses. Their strategy utilizes a QuilA-loaded chitosan (QAC)-encapsulated plasmid DNA (pQAC) vaccine to prime the immune system, followed by a modified vaccinia Ankara (MVA) vaccine boost. Both the pQAC plasmids (pQAC-CoV) and MVA vector (MVA-CoV) were designed to express spike (S) and nucleocapsid (N) antigens derived from a strain of SARS-CoV-2 from the early phase of the COVID-19 pandemic. This prime/boost (PB) strategy is referred to herein as either “pQAC/MVA-CoV” or “the heterologous vaccine.” The inventors' results indicate that intranasal administration of pQAC/MVA-CoV induces robust systemic and local neutralizing antibodies in mice. Importantly, the humoral responses of these mice were complemented by the induction of localized Th17 cellular responses. In addition, mice that were vaccinated with three doses of only plasmid vector (pQAC-CoV) via either the intranasal or the intramuscular route generated robust type 1 and type 17 (Tc17 or Th17) cellular responses. Importantly, while the heterologous vaccine generated a robust immune response, it did not produce danger signals indicative of a cytokine storm, suggesting that it could be relatively safe.

### Compositions:

In a first aspect, the present invention provides DNA vaccine compositions. These vaccine compositions comprise an adjuvant and a polynucleotide that encodes a SARs-CoV-2 spike (S) protein, a SARs-CoV-2 nucleocapsid (N) protein, or both the S protein and the N protein. The proteins need not be full length proteins. As shown in the examples, a portion of the S protein truncated to not included the transmembrane portion of the protein and only include the extracellular portion was sufficient to elicit an immune response. The adjuvant used in the Examples comprises disaggregated spherical nanostructures comprising Quil-A and chitosan, which are present at a ratio between 1:15 and 1:100.

As used herein, the terms “DNA vaccine,” “nucleic acid vaccine,” and “plasmid vaccine” are used interchangeably refer to a polynucleotide encoding at least one antigen. Following immunization, a subject's cells take up the polynucleotide and express the encoded antigen from it, inducing an immune response against the antigen. DNA vaccines offer several potential advantages over traditional vaccine strategies, including the stimulation of both B- and T-cell responses, improved storage stability, the absence of any infectious agent, and the relative ease of large-scale manufacture. However, DNA vaccines also come with several challenges, including in vivo degradation of the construct by DNases, inefficient uptake by antigen presenting cells, and low immunogenicity (9, 10). The inventors envision that the use of articulate delivery systems, such as QuilA-loaded Chitosan (QAC) nanoparticles used with the present invention, may overcome these challenges by facilitating a prolonged release of active plasmid (11). Nucleic acid-based vaccines generally contain additional elements in addition to the polynucleotide encoding the antigen such as a promoter functional in cells of the subject to be immunized, or may be altered to offer increased stability or resistance to degradation in the host cell.

As used herein, “antigen” refers to a substance that induces a targeted immune response in a subject. For example, in some embodiments, the compositions disclosed herein comprise one or more polynucleotides that encode a SARS-CoV-2 spike (S) protein, a SARS-CoV-2 nucleocapsid (N) protein, or both the S protein and the N protein. Therefore, in the foregoing example, the antigens are the SARS-CoV-2 S and N proteins that are encoded by the one or more polynucleotides. In some embodiments, the S proteins are encoded by one or more of the group consisting of SEQ ID NOs:1 and 3. The S proteins encoded by these polynucleotides are provided as SEQ ID NOs: 2 and 4, respectively and any polynucleotide encoding SEQ ID NO: 2 or 4 is included as the coding sequence for the proteins may be optimized for expression in particular cell types. In some embodiments, the N proteins are encoded by SEQ ID NO: 5. The N proteins encoded by this polynucleotide are provided as SEQ ID NO: 6 and any polynucleotide encoding SEQ ID NO: 6 is also encompassed herein. The polynucleotides provided herein may be altered to optimize codon usage for maximal expression in a particular host such as a human subject. Thus, the sequences provided herein also include sequences with 70%, 75%, 80%, 85%, 90%, 92%, 94%, 95%, 96%, 97%, 98%, or 99% identity to the sequences of SEQ ID NO: 1, 3 or 5. The proteins encoded by the polynucleotides may also encompass changes especially as these proteins are known to exist in various isoforms and be antigenically diverse in strains or variants of SARS-CoV-2. The sequences provided herein also include sequences with 70%, 75%, 80%, 85%, 90%, 92%, 94%, 95%, 96%, 97%, 98%, or 99% identity to the amino acid sequences of SEQ ID NO: 2, 4 or 6. In some embodiments, the polynucleotide encodes both the S and N proteins on a single molecule. As such, in some embodiments, the polynucleotide comprises sequences linking the S and N proteins. The N and S sequences may be linked via a polynucleotide of any length but should be in frame or contain independent regulatory regions such as an internal ribosome entry site to allow for expression of both proteins from the polynucleotide.

As used herein, a “fragment” is a portion of an amino acid sequence which is identical in sequence to, but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. A fragment may include an N-terminal truncation, a C-terminal truncation, or both N-terminal and C-terminal truncations relative to the full-length reference polypeptide. The truncated S included here is an example of a fragment and lacks the transmembrane domain of the S protein to allow for better expression and soluble fragments of S protein to be generated.

The term “recombinant” when used with reference, e.g., to a cell, or nucleic acid, protein, expression cassette, or vector, indicates that the cell, nucleic acid, protein, expression cassette, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed, or not expressed at all.

The polynucleotide vaccine compositions provided herein may be DNA or RNA and may include regulatory regions to allow for transcription and/or translation of the polynucleotides into polypeptides once in a cell of a vaccinated subject. The polynucleotides may be operably linked to promoters functional in cells of the vaccinated subject. The polynucleotides may be altered to allow for resistance to nucleases.

However, as discussed above, DNA vaccines can suffer from several drawbacks including in vivo degradation of the construct by DNases, inefficient uptake by antigen presenting cells, and low immunogenicity. In some embodiments, the vaccine composition further comprises an adjuvant. As used herein, the term “adjuvant” or “vaccine adjuvant” refers to any substance that non-specifically enhances the immune response to an antigen. The adjuvant used with the vaccine compositions disclosed herein in the Examples is a Quil-A chitosan (QAC) complex, in which Quil-A and chitosan are combined to form distinct disaggregated spherical nanostructures. The QAC complexes are loaded with one or more payload molecules (in this case, the antigen-encoding polynucleotide) with which the QAC complex stimulates an immune response. The QAC complex adjuvant was previously described in International Application No. PCT/US2020/037438 (International Publication No. WO2020/252263), and Chandrasekar et al. 2020 A Novel Mucosal Adjuvant System for Immunization Against Avian Coronavirus Causing Infectious Bronchitis J Virol 94:e01016-20, which are both hereby incorporated by reference in their entirety. Advantageously, QAC-adjuvanted vaccines appear to target local mucosal immunity, which should result in a more effective immune response to SARS-CoV-2 given that airway epithelium T cells and IgA humoral responses have been shown to be critical for restricting respiratory viral pathogens like SARS- and MERS-CoV (12).

“Quil-A” refers to the powdered saponin fraction isolated from extract of the bark of Quillaja saponaria trees. Quil-A is commercially available, for example from Desert King sold under the product name Vet-Sap™.

“Chitosan” refers to a linear polysaccharide composed of randomly distributed β-linked D-glucosamine and N-acetyl-D-glucosamine. Chitosan can be obtained from the chitin shells of shrimp and other crustaceans by treatment of the shells with an alkaline substance. Chitosan is a non-toxic, naturally occurring cationic polymer that readily complexes with DNA and negatively charged proteins. Chitosan is biocompatible and biodegradable. Compositions incorporating chitosan have sustained release kinetics and are immunomodulary, enhancing the T-cell response. In some embodiments, chitosan is deacetylated chitosan, for example deacetylated chitosan (>75%). Deacetylated chitosan is available commercially from Sigma (C3646). Higher deacetylation percentages, for example about 90%, will meditate stronger binding with nucleic acids resulting in slower release kinetics from the nanoparticle structures of the QAC complex. In some embodiments, the chitosan is at least 70%, 75%, 80%, 85%, 90%, or 95% deacetylated. In some embodiments, the chitosan is between about 60% and about 90% deacetylated.

In some embodiments, the chitosan is functionalized. Chitosan may be functionalized with negatively charged sulfonate groups by reaction of the amino group of chitosan with 5-formyl-2-furan sulfonic acid (FFSA) followed by treatment using sodium borohydride to form a negatively charged chitosan surface. Use of the negatively charged chitosan in the formation of the QAC complex will generally be favorable for loading of positively charged payload molecules.

The QAC complex is loaded with the antigen-encoding polynucleotide by mixing a solution of Quil-A and polynucleotide into a solution of chitosan to form a final mixed solution containing a QAC-polynucleotide complex. In the final mixed solution, the Quil-A and the chitosan are present at a ratio of between 1:15 to 1:100. In some embodiments, the Quil-A and the chitosan are present at a ratio of about 1:20 (e.g., 1:15, 1:16, 1:17, 1:18, 1:19, 1:20, 1:21, 1:22, 1:23, 1:24, or 1:25) in the final mixed solution. In some embodiments, in the final solution Quil-A is at a concentration of 0.001% and chitosan is at a concentration between about 0.02% and about 0.1%.

The QAC complex nanostructures are less 100 nm in diameter when measured in the absence of any payload molecules. For example, the nanostructures may be between about 5 nm and about 100 nm, between about 10 nm and about 95 nm, between about 15 nm an about 90 nm, between about 20 nm and about 90 nm, or between about 25 nm and about 85 nm in the absence of a payload molecule. The QAC complex may be loaded with one or more payload molecules such as the polynucleotides described herein encoding a SARs-CoV-2 spike (S) protein, a SARs-CoV-2 nucleocapsid (N) protein, or both the S protein and the N protein. The nucleotide-QAC complex may be between about 20 nm and about 1000 nm in diameter. The specific size of the nucleotide-QAC complex will vary depending on the size and amount of payload in the nanostructure. As used herein, “disaggregated,” refers to the formation of discrete observable particles as opposed to aggregated non-discrete assemblies with non-distinct boundaries and “spherical” means roughly spherical in nature and is not meant to be a precise definition of the structure.

Though the QAC adjuvant strategy significantly improves the immunogenicity and protective immune response generated by the DNA vaccine compositions of the current disclosure, the inventors hypothesized that a heterologous vaccine approach may further increase the effectiveness of the compositions. As used herein, “heterologous vaccine approach” refers to practice of inducing a first immune response with a first vaccine composition, then inducing a second immune response with a second different vaccine composition. Accordingly, a “heterologous vaccine” may also refer to the “second different vaccine composition” in the preceding example.

In a second aspect, the present invention provides viral vaccine compositions. These vaccine compositions comprise a viral vector that comprises a polynucleotide encoding a SARs-CoV-2 spike (S) protein, a SARs-CoV-2 nucleocapsid (N) protein, or both the S protein and the N protein or portions thereof. The antigens and polynucleotides encoding the antigens as described above for the polynucleotide-based vaccine vector may also be incorporated into the viral vaccine compositions described herein.

As used herein, a “viral vector” refers to a virus or viral particle that comprises a polynucleotide encoding at least one antigen. The viral vector delivers the polynucleotide into a vaccinated subject's cells. Within the cell, the polynucleotide is transcribed and translated, producing the encoded antigen and inducing an immune response against it. The viral vectors of the present invention are “recombinant viruses,” in which foreign genetic material encoding an antigenic protein (i.e., from SARS-CoV-2) has been inserted into the viral genome.

The viral vectors may be a weakened or killed version of a virus. For example, the viral vector can be based on an attenuated virus, which does not replicate or exhibits very little replication in a host but is able to introduce and express a foreign gene in infected cells. As used herein, an “attenuated virus” is a strain of a virus whose pathogenicity has been reduced compared to its natural counterpart. A virus may be attenuated using serial passaging, plaque purification, or other means.

In some embodiments, the viral vector is selected from an adeno-associated virus or a poxvirus. Suitable poxviruses for use with the present invention include, without limitation, canary poxvirus, raccoon poxvirus, vaccinia virus, fowl poxvirus and myxoma virus (MYXV). Poxviruses are a preferred choice for transferring genetic material into new hosts due to their relatively large genome size (approximately 150-200 kb) and because of their ability to replicate in the infected cell's cytoplasm rather than the nucleus, thereby minimizing the risk of integrating genetic material into the genome of the host cell. Of the poxviruses, the vaccinia and variola species are the two best known. Vaccinia virus is highly immunogenic, provoking strong B-cell (humoral) and T-cell mediated immune responses against its encoded gene products. Of these viruses, the modified vaccinia virus Ankara (MVA) is particularly safe, as it has diminished virulence while maintaining good immunogenicity. Thus, in preferred embodiments, the viral vector is a modified vaccinia Ankara (MVA) virus. Exemplary MVA virus strains include MVA 572, MVA 575, and MVA-BN, which have been deposited at the European Collection of Animal Cell Cultures (ECACC), Salisbury (UK) with the deposition numbers ECACC V94012707, ECACC V00120707 and ECACC V00083008, respectively, and are described in U.S. Pat. Nos. 7,094,412 and 7,189,536.

Both the DNA vaccine compositions and the viral vaccine compositions of the present invention comprise a polynucleotide encoding a SARs-CoV-2 spike (S) protein, a SARs-CoV-2 nucleocapsid (N) protein, or both the S protein and the N protein. The S protein is a surface-exposed protein that mediates SARS-CoV-2 entry into cells via its interaction with the ACE2 receptor, while the N protein is a multifunctional RNA-binding protein necessary for viral RNA transcription and replication. Both of these proteins are promising targets for SARS-CoV-2 vaccines. In some embodiments, the vaccines encode a full-length S protein (nucleotide sequence: SEQ ID NO:1; amino acid sequence: SEQ ID NO:2). However, the inventors have determined that removing the transmembrane domain of this protein makes it easier to express by enabling secretion of S protein needed for a robust humoral response. Thus, in other embodiments, the vaccines encode a truncated S protein lacking the transmembrane domain (TrS; nucleotide sequence: SEQ ID NO:3; amino acid sequence: SEQ ID NO:4). In some embodiments, vaccines encode the full-length N protein (nucleotide sequence: SEQ ID NO:5; amino acid sequence: SEQ ID NO:6). The nucleotide sequences presented herein for the TrS and N proteins are modified from the native sequences to optimize the codon usage for human expression. Thus SEQ ID NO: 3 and SEQ ID NO: 5 represent non-native nucleotide sequences for the TrS and N proteins, respectively.

In the Examples, the inventors immunized mice with vaccines that expressed both the truncated S protein and the full-length N protein. Inclusion of both N and S proteins elicits a well-rounded immune response with induction of both humoral (S protein) and cell-mediated immune responses (S and N protein). Thus, in some embodiments, the polynucleotide encodes both the S protein and the N protein, and in some embodiments, the polynucleotide comprises both SEQ ID NO:3 and SEQ ID NO:5 (i.e., the sequences encoding the truncated S protein and the full-length N-protein, respectively). In the vaccine compositions these polynucleotides may be operably linked to promoters functional in the targeted cells such that when the vaccine composition gains entry into a cell the polynucleotides express the proteins of SEQ ID NO: 2, 4 or 6. The proteins may also be present on the viral vaccine compositions. SARS-CoV-2 variants have already been identified and these variants are also encompassed. The vaccine compositions may include polynucleotides having 85%, 90%, 92%, 94%, 95%, 96%, 97%, 98% or 99% identity to SEQ ID NO: 1, 3 and 5. The vaccine compositions may include or having polynucleotides encoding for proteins having 85%, 90%, 92%, 94%, 95%, 96%, 97%, 98% or 99% identity to SEQ ID NO: 2, 4 and 6.

The vaccine compositions of the present invention may be used as a prophylactic, e.g., to prevent or ameliorate the effects of a future infection by SARS-CoV-2, or may be used as a therapeutic, e.g., to treat COVID-19. The vaccines provided herein are expected to induce and enhance the immune response of the subject to SARS-CoV-2. The immune response enhanced is suitably a polyfunctional response.

The vaccine compositions may further comprise other suitable agents or ingredients. Suitable agents may include a suitable carrier or vehicle for delivery. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories may be found in the U.S. Pharmacopeia National Formulary, 1857-1859, (1990).

The vaccine formulation may be separated into vials or other suitable containers. The vaccine formulation herein described may then be packaged in individual or multi-dose ampoules or be subsequently lyophilized (freeze-dried) before packaging in individual or multi-dose ampoules. The vaccine formulation herein contemplated also includes the lyophilized version. The lyophilized vaccine formulation may be stored for extended periods of time without loss of viability at ambient temperatures. The lyophilized vaccine may be reconstituted by the end user and administered to a patient.

### Methods:

The present invention also provides methods of inducing an immune response against SARs-CoV-2 in a subject. The methods comprise administering one of the nucleic acid-adjuvant vaccine compositions or viral vaccine compositions disclosed herein in an amount effective to induce the immune response against at least one SARS-CoV-2 antigen in the subject.

In the present methods, the vaccine compositions may be administered to the subject by an oral, oronasal, intranasal, parenteral or intramuscular route. Currently, most experimental DNA vaccines are only amenable for intramuscular administration, which produces only limited mucosal immunity. However, the inventors envision that mucosal vaccination strategies (i.e., intranasal vaccinations) may be more effective against COVID-19, as local mucosal immune responses have been shown to be critical for restricting other respiratory viral pathogens like SARS, MERS CoV, etc. (22, 23, 29, 30). Intranasal administration offers several additional advantages, e.g., it is noninvasive, causes little discomfort to patients, and can be performed by someone with minimal training. While intranasal vaccinations can be challenging, owing to the low bioavailability, antigen uptake, and antigen degradation at mucosal surfaces (31, 32), the inventors believe that inclusion of the QAC complex adjuvant with an intranasal vaccine will circumvent some of these challenges (11). Intranasal administrations also mediate robust induction of local mucosal responses that are important for controlling respiratory pathogens. Thus, in some embodiments, the administration is by an intranasal route. In other embodiments, the administration is by an intramuscular route.

As used herein, “subject” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, such as chickens and other poultry. The term “subject” does not denote a particular age or sex. In one embodiment, the subject is a human. In a preferred embodiment, the human is at risk of being infected with SARs-CoV-2.

The phrase “amount effective to induce the immune response,” as used herein, refers to an amount of a vaccine composition that would induce a humoral immune response against at least one SARS-CoV-2 antigen (e.g., the spike or nucleocapsid protein encoded by the disclosed vaccines) and suitably also induces a polyfunctional T cell response as well. Humoral immunity or cell mediated immunity or both humoral and cell mediated immunity may be induced. The immunogenic response of an animal to a vaccine may be evaluated, e.g., indirectly through measurement of antibody titers, lymphocyte proliferation assays, or directly through monitoring signs and symptoms after challenge with the virus. The protective immunity conferred by a vaccine may also be evaluated by measuring, e.g., clinical signs such as mortality, morbidity, temperature, overall physical condition, overall health, weight of the subject, and the performance of the subject. The amount of a vaccine that is therapeutically effective may vary depending on the particular strain of virus used, the antigen used in the vaccine, the species of the subject, the condition of the subject (e.g., age, body weight, gender, health), and should be determined by a physician. The therapeutically effective amount may be administered in one or more doses and is preferably in the range of about 0.01-10 mL, most preferably 0.05-1 mL, containing 1-200 micrograms, most preferably 1-100 micrograms of vaccine formulation/dose.

In another aspect, the present invention provides methods of inducing an immune response against a SARs-CoV-2 in a subject using a heterologous immunization strategy. The methods comprise (a) administering a first vaccine composition comprising one of the DNA vaccine compositions or viral vaccine compositions disclosed herein, and (b) administering a second vaccine composition comprising one of the DNA vaccine compositions or viral vaccine compositions disclosed herein, wherein administration of the first vaccine composition and the second vaccine composition induces the immune response against at least one SARS-CoV-2 antigen in the subject.

In some embodiments, administration of the second vaccine composition occurs at least three weeks after administration of the first vaccine composition. However, in the Examples the inventors detected the greatest immune response when the viral vaccine was administered six weeks after the QAC-adjuvanted DNA vaccine. Thus, in some embodiments, administration of the second vaccine composition occurs at least six weeks after administration of the first vaccine composition. A hallmark of the QAC adjuvant system is slow-release of payload with continual priming of the immune system. We hypothesize that release of DNA payload can be sustained up to six weeks after which another immunization will further boost immune responses.

In some embodiments, both the first vaccine composition and the second vaccine composition are DNA vaccine compositions disclosed herein. In other embodiments, the first vaccine composition is a DNA vaccine composition disclosed herein and the second vaccine composition is a viral vaccine composition disclosed herein. The DNA/DNA vaccine resulted in the induction of robust local CD8+ T-cells that secrete IFNγ, TNFα or IL-2 without a complementing humoral response. On the other hand, the DNA/MVA vaccine resulted in the induction of both local and systemic T-cell (Th17) and humoral responses. The distinct type of immune response induced by the DNA vaccine followed by the viral vectored vaccine was unexpected and suggests this form of vaccination may have advantages in limiting spread of the virus from lungs to other sequelae of infection.

In the heterologous immunization methods, the two vaccines may be administered by an intranasal route or by an intramuscular route. Specifically, in some embodiments, both the first vaccine composition and the second vaccine composition are administered by an intranasal route. In other embodiments, both the first vaccine composition and the second vaccine composition are administered by an intramuscular route. In other embodiments, the first vaccine composition is administered by an intranasal route and the second vaccine composition is administered by an intramuscular route. In other embodiments, the first vaccine composition is administered by an intramuscular route and the second vaccine composition is administered by an intranasal route. As shown in the examples, some routes or combinations of routes of infection resulted in changes in immune responsiveness and may be recommended for distinct populations of subjects.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

The rapid transmission of SARS-CoV-2 in the USA and Worldwide necessitates the development of multiple vaccines to combat the COVID-19 global pandemic. Previously, we showed that a novel adjuvant system, QuilA-loaded Chitosan (QAC) nanoparticles, can elicit robust immunity combined with plasmid vaccines when used against avian coronavirus. Here, we report a prime/boost immunization using a plasmid vaccine and a modified vaccinia Ankara (MVA) expressing the SARS-CoV-2 Spike (S) and Nucleocapsid (N) antigens. Only the heterologous intranasal immunization strategy elicited robust neutralizing antibodies against SARS-CoV-2 in serum and bronchoalveolar lavage of mice, suggesting a protective vaccine. The same prime/boost strategy led to the induction of type 1 and type 17 T-cell responses and polyfunctional T-cells expressing multiple type 1 cytokines (e.g., IFN-γ, TNFα, IL-2) in the lungs and spleens of vaccinated mice. The outcomes of this study support further development of QAC-nano vaccines to control the COVID-19 pandemic.

### Example 1

### Materials and Methods:

Ethics Statement

All animals used in this study were cared for per established guidelines and experimental protocols approved by the Institutional Animal Care and Use Committee (IACUC) of the University of Wisconsin-Madison.

Cell Lines

HEK 293T and Vero E6 cells were a kind gift from Dr. Jorge Osorio. Chicken Embryonic Fibroblasts (CEF) were prepared from 9-day-old specific pathogen-free (SPF) white leghorn eggs (Charles River Laboratories, Inc., WA, USA) as described previously (47). Human Embryonic Kidney Cells (HEK-293T) expressing human angiotensin-converting enzyme 2 (ACE2), HEK-293T-hACE2 Cell Line, NR-52511 was obtained through BEI Resources, NIAID, NIH. All cells were maintained in DMEM supplemented with 10% fetal bovine serum (FBS) and penicillin-streptomycin (D10) at 37° C., 5% CO₂ atmosphere.

Preparation of SARS-CoV-2 Vaccine Constructs

Sequences for the SARS-CoV-2 spike (S) and nucleocapsid (N) genes were downloaded (GenBank accession number MN908947), back-translated, and codon-optimized for expression in mice. DNA fragments encoding truncated S with the trans-membrane domain deleted (TrS) and N antigens were commercially synthesized and cloned into a pCMV backbone expression vector with a C-terminus 6×His tag (Twist Bioscience, CA, USA). To confirm the insertion of genes in the correct orientation, DNA sequencing was performed at the UW-Madison Biotechnology Center with an ABI Prism 3730XL DNA analyzer using BigDye terminators (Applied Biosystems, CA). To confirm the expression of TrS and N proteins, HEK 293T cells seeded in 6-well format were transfected with an optimized ratio of DNA (3 ug): FuGENE HD (9 μl) according to manufacturer's instructions (Promega, WI, USA). Three days post-transfection, cells, and supernatant (separately) were harvested for western blot analysis. The MVA expressing N and TrS constructs were generated in CEF cells as described previously (48). The cell and supernatant fractions were boiled in Laemmli sample buffer (BioRad, Hercules, Calif., USA) and resolved on a 4-20% SDS-PAGE gel by electrophoresis using a Mini-PROTEAN 3 system (BIO-RAD, CA). Polyacrylamide gels were electroblotted onto nitrocellulose membranes using a Turboblot® system. Membranes were blocked in 5% (W/V) skim milk and probed with Direct-Blot™ HRP anti-6-His Epitope Tag Antibody (BioLegend, #906109) or polyclonal mouse anti-SARS-CoV-2 spike sera harvested from one pQAC/MVA-CoV immunized C57BL/6 mouse three weeks post final boost. Membranes were developed using a solid phase 3, 30, 5, 50-tetramethylbenzidine (TMB) substrate system. Plasmid loaded QAC particles were synthesized as described previously (11).

Vaccine Efficacy Study

The immunogenicity of the experimental vaccine constructs was evaluated in C57BL/6 mice (6 weeks of age) obtained from Taconic Inc. and maintained in bio-safety level-2 containment. At every indicated time point, mice were concurrently immunized with MVA or pQAC TrS and N constructs. A total of 50 mice were divided equally into five groups (n=10 each). Groups of mice were either unvaccinated (PBS), immunized with pQAC-CoV (I.M), or immunized with pQAC-CoV (I.N) at week-0, week-3, and week-6. Another group of C57BL/5 mice was vaccinated with pQAC-CoV (I.N) at week-0, followed by a boost with MVA-CoV (I.N) at week-6. A vaccine dose of 75 μg/plasmid DNA construct/animal, and 10⁸ pfu/MVA construct/animal was administered at each immunization time point. Sera for ELISA and neutralizing antibody titers were harvested from blood collected at week-6 and -9. At week-6, three weeks post first boost, and at week-9, three weeks post final boost, mice (n=5 each time-point) were euthanized, BAL was collected in D10 media as previously described (49), and lungs and spleen were harvested and processed for ICS assay as described below.

SARS-CoV-2 Specific ELISA

Sera and BAL from different timepoints were screened for humoral response against SARS-CoV-2 Spike. To measure IgG and IgA antibody levels in plasma and BAL of mice, a SARS-CoV-2 specific enzyme-linked immunosorbent assay (ELISA) was developed. Briefly, Nunc ELISA plates were coated with SARS-CoV-2 spike protein (BEI resources-NR-52396, 100 ng total/well) diluted in carbonate/bicarbonate buffer, pH 9.6, and incubated overnight at 4° C. followed by blocking with 5% Skim milk to reduce background. A total of 100 μl of diluted serum (1/25) or BAL (undiluted) harvested at different time-points from immunized mice was added to the wells and incubated at 37° C. for 1 hr. Post washing (PBS-TritonX 100, 0.1%), either HRP conjugated anti-mouse IgG (1036-05, Southern Biotech) or anti-mouse IgA (1040-05, Southern Biotech) was added to the wells at dilutions of 1/1000 and incubated at 37° C. for 1 hr. Post washing, 100 μl of TMB substrate solution was added and incubated for 20 min or until color developed. The addition of 1M sulphuric acid stopped the reaction, and plates are read at 450 nm. Binding antibody EPTs were calculated as described previously (15).

SARS-CoV-2 Pseudovirus Neutralization Assay

SARS-CoV-2 pseudotyped virus based on an HIV-pseudotyped luciferase-reporter based system was generated as described elsewhere and was used to perform the neutralization assay (25). The SARS-Related Coronavirus 2, Wuhan-Hu-1 Spike-Pseudotyped Lentiviral Kit, NR-52948, was obtained through BEI Resources, NIAID, NIH. For the neutralization assay, heat-inactivated sera were first serially diluted and incubated with the virus for 90 min at 37° C. Then the serum-virus mixture was transferred into wells pre-seeded with HEK293T-hACE2 cells (BEI resources NR-52511). After 48 hr, cells were lysed, and luciferase activity was measured using the ONE-Glo™ Luciferase Assay System (Promega, WI, USA). RLU were measured using the TD 20/20 Luminometer (Turner Designs, CA, USA). Neutralization titers (ID₅₀) were calculated as the serum dilution at which RLU were reduced by 50% compared with RLU in virus control wells after subtraction of background RLU in cell control wells.

SARS-CoV-2 Wild-Type Virus Neutralization Assay

SARS-CoV-2, Isolate USA-WA1/2020, a kind gift from Dr. Jorge Osorio, was propagated and titrated on Vero E6 cells. Human plasma samples were obtained through BEI Resources, NIAID, NIH: Human Plasma, Sample ID WU353-108, NR-53675-53679, contributed by Ali Ellebedy, Ph.D., Washington University School of Medicine, St. Louis, Mo., USA. Heat-inactivated sera and BAL were first serially diluted in serum-free Opti-MEM media and incubated with 100 PFU per well of SARS-CoV-2, Isolate USA-WA1/2020 for 60 min at 37° C. and transferred into wells pre-seeded with Vero E6 cells. Serum and BAL dilutions were performed in duplicate. Plates were incubated at 37° C. for four days before scoring for the cytopathic effect. Neutralization titer was calculated as the reciprocal of the highest dilution at which virus neutralization occurred.

Flow Cytometric Assessment of SARS-CoV-2 Specific Intracellular Cytokine Assay

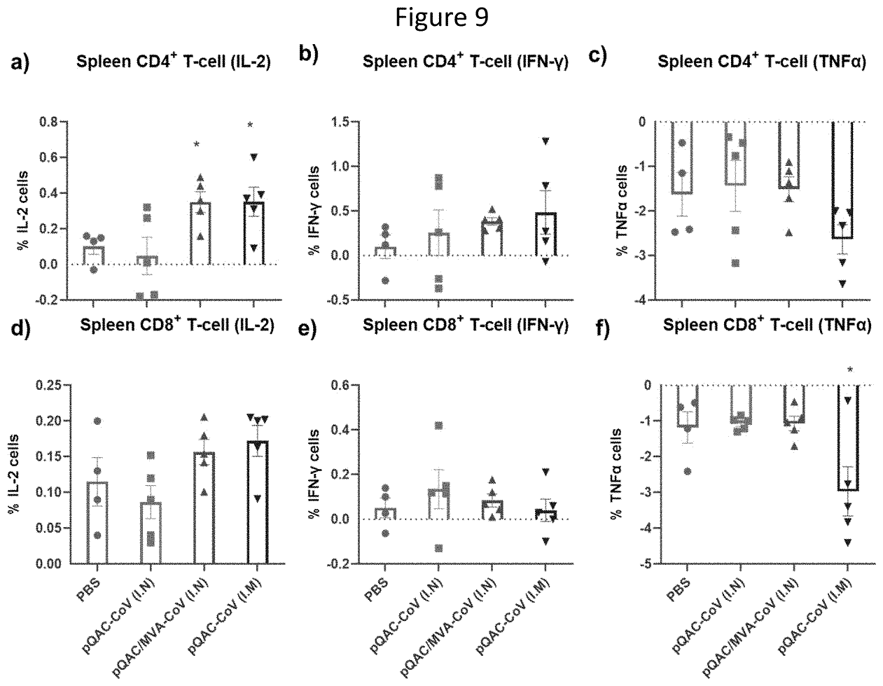

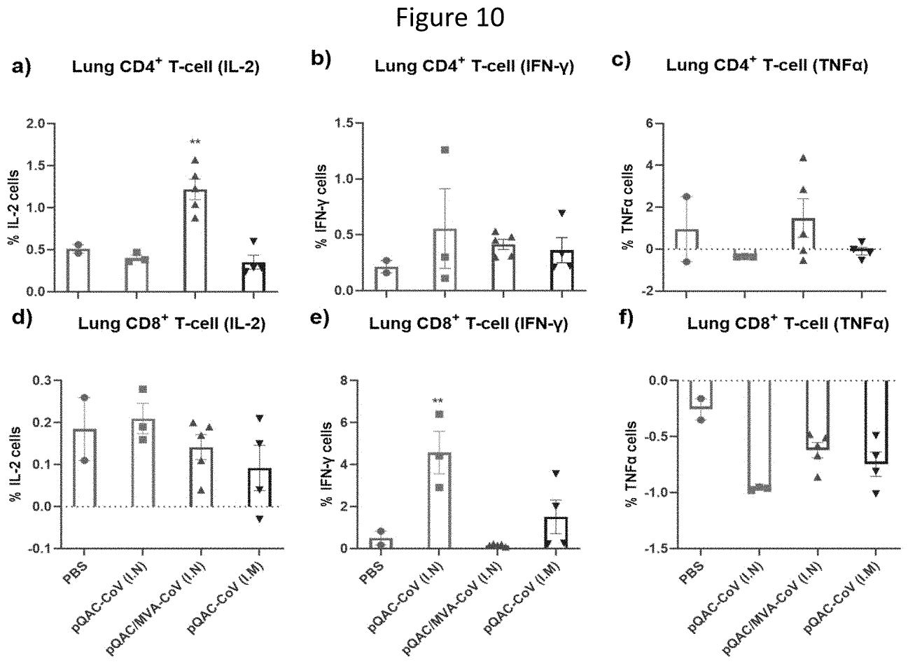

Immunized C57BL/6 mice (N=5) from each vaccine group were euthanized 3 weeks post final boost and used for flow cytometric assessment. Single-cell suspensions from the lungs and spleen were prepared using standard techniques. Briefly, lungs were excised and placed in a gentleMACS dissociator M tube (Miltenyi 130-093-236) with 3 mL collagenase B (1 mg/ml, Roche). Lung tissue was processed using the gentle MACS dissociator, followed by incubation for 30 min at 37° C. Single-cell suspensions lung and spleen were prepared by gently squeezing through a 70 mm cell strainer (Falcon) after lysing RBCs using 1×BD Biosciences BD Pharm Lyse™. For intracellular cytokine staining, 1×10⁶ cells were stimulated with SARS-CoV-2 spike protein (BEI resources-NR-52396, 100 ng total/well) overnight (˜18 hr) at 37° C. Brefeldin A (1 μl/ml, GolgiPlug, BD Biosciences) was added and cells were further incubated for another 5 hr at 37° C. Fluorochrome-labeled antibodies against the cell-surface antigens CD4 (BUV 496, GK1.5), CD8a (BUV395, 53-6.7) and intracellular antigens IFN-γ (APC, XMG1.2), TNF-α (BV421, MP6-XT22), IL-2 (PE-CF594, JES6-5H4), IL-17 (FITC, TC11-18H10.1), IL-13 (PE-Cy7, eBio13A) were purchased from BD Biosciences (San Jose, Calif.), Biolegend (San Diego, Calif.), eBioscience (San Diego, Calif.), or Invitrogen (Grand Island, N.Y.). Before antibody staining, cells were stained for viability with Dye eFluor 780 (eBiosciences, San Diego, Calif.). After stimulation, cells were stained for surface markers and then processed with the Cytofix/Cytoperm kit (BD Biosciences, NJ). To stain for cytokines, cells were first stained for cell surface molecules, fixed, permeabilized, and then subsequently stained for the cytokines. All samples were acquired on an LSR Fortessa (BD Biosciences) flow cytometer. Data were analyzed with FlowJo software (TreeStar, OR). Results are expressed as the difference between the percentage of stimulated cells and the percentage of unstimulated cells. A Boolean gating strategy was applied for the determination of cytokine secreting T cells (See FIG. 8 for gating strategy and FIGS. 9-10 for single cytokine staining).

Statistical Analysis

Statistical analyses were performed using GraphPad software (La Jolla, Calif.). ELISA EPT and BAL nAb titers were compared using a student's t-test where *, P<0.05; **, P<0.01 were considered significantly different among groups. Serum neutralizing antibody titers and cellular immune assays were compared using an ordinary one-way ANOVA test where *, P<0.05; **, P<0.01 were considered significantly different among groups. Antibody titers (ELISA) were compared using a two-way ANOVA test where *, P<0.05; **, P<0.01 were considered significantly different.

### Results:

Design and Construction of SARS-CoV-2 Vaccine Constructs

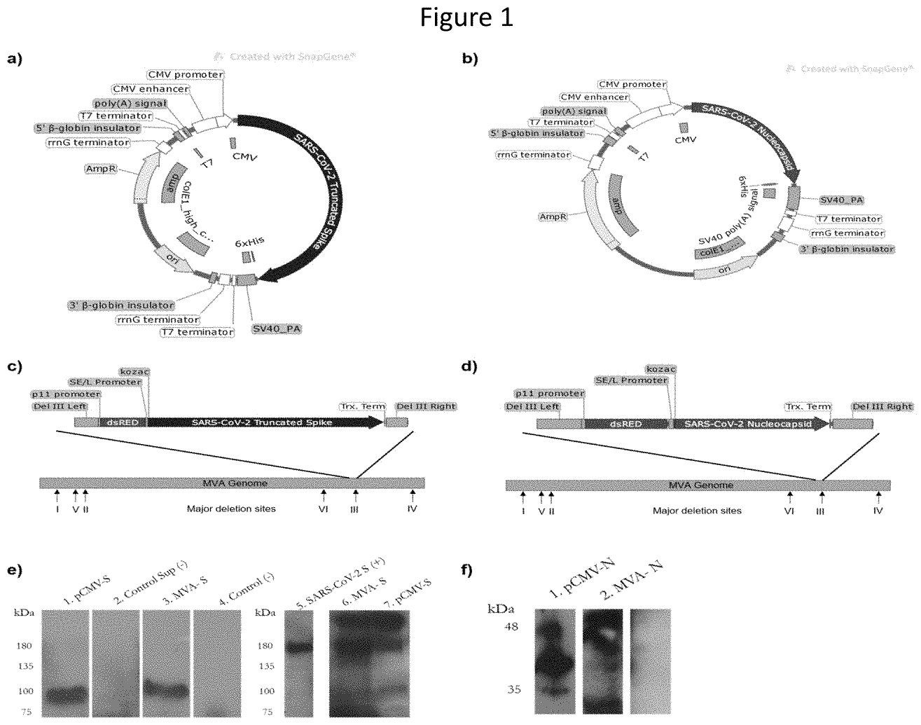

Plasmid DNA (pDNA) vaccine candidates encoding the spike (S) gene of SARS-CoV-2 with a deletion of the transmembrane domain (TrS) and nucleocapsid gene (N) with the addition of C-terminal 6×His tag were independently constructed. The genes from the first sequenced Wuhan SARS-CoV-2 isolate (GenBank accession number MN908947) were used for constructing the vaccine candidates. The expression of transgenes from the plasmid DNA vaccine candidates was confirmed using western blot analysis on supernatant and cells harvested from transfected HEK-293T cells with an anti-6×His antibody (FIGS. 1a and b). The same genes were cloned into a modified vaccinia Ankara (MVA) shuttle vector via homologous recombination with and insertion into the deletion III region within the MVA genome (FIGS. 1c and 1d). The TrS and N protein expression in MVA vaccine candidates are under the control of the SE/L promoter. Western blot analysis confirmed protein expression in supernatant from MVA-TrS and cell pellets from MVA-N infected CEF cells (FIGS. 1e and 1f). For TrS, a single band of about 90 kDa was detected by the anti-6×His antibody, reflecting the proteolytic cleavage of the secreted protein (FIG. 1e). Three major bands, around 250 kDa, 180 kDa, and 90 kDa, were observed when mouse sera that was reactive to SARS-CoV-2 spike protein was used for western blot analysis. These bands indicate the presence of both full-length and cleaved secreted spike proteins from the plasmid DNA and MVA vaccine candidates (FIG. 1e).

Induction of SARS-CoV-2 Specific Humoral Responses in Vaccinated Mice

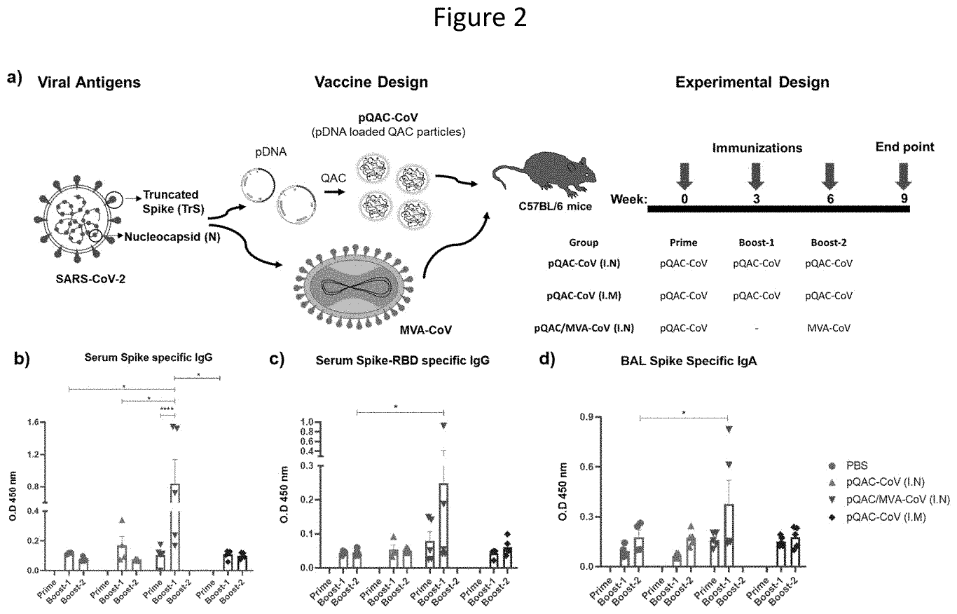

Previously, we demonstrated that a 2-dose QAC-encapsulated plasmid DNA (pQAC) encoding the nucleocapsid gene of avian coronavirus provided protection that was equivalent to that of live attenuated vaccines when birds were challenged with infectious bronchitis virus (11). However, only T-cell responses without a complementing humoral response were observed in the vaccinated birds. Based on these results, we hypothesized that a heterologous strategy of priming with plasmid encoded-antigens and boosting with MVA expressing the same antigens would overcome this limitation and elicit both robust T-cell and humoral immune responses (20, 21). To test this hypothesis, groups of C57BL/6 mice were initially immunized with QAC complexed plasmid DNA (pCMV-TrS and pCMV-N, both termed pQAC-CoV) via intranasal (I.N) or intramuscular (I.M) routes followed by two additional doses at 3 and 6 weeks post initial immunization (75 μg plasmid DNA/dose/mouse) for a total of 3 doses. Another group of C57BL/6 mice was initially immunized with pQAC-CoV followed by a boost with MVA-CoV at week 6, both via the I.N. route, for a total of 2 doses. Interestingly, 3 weeks after a prime and one boost immunization, S-specific and S receptor-binding domain (RBD)-specific IgG were detected in the sera from the heterologous vaccine group (pQAC/MVA-CoV) at levels that were significantly higher than the rest of experimental groups (FIGS. 2a and 2b). The antibody levels above the baseline for the PBS-immunized group were non-significant for both the pQAC-CoV I.N and I.M groups (FIGS. 2a and 2b). A single-dose administration of pQAC-CoV (heterologous group, prime timepoint) also elicited detectable S- and RBD-specific IgG levels that were higher than observed in the PBS group (FIGS. 2a and 2b).

Finally, we also examined mucosal secretory IgA (sIgA) produced in the upper respiratory tract that is critical in limiting infection of many respiratory pathogens, including coronaviruses at the primary site of infection (22-24). The S-specific IgA (IgA) levels in the bronchoalveolar lavage (BAL) of immunized mice in the heterologous vaccine group were significantly higher than those of the PBS group (FIG. 2c). However, we did not detect a significant induction of sIgA in any plasmid immunized mice, even after prime and two boost doses. The presence of sIgA and circulating IgG in pQAC/MVA-CoV immunized mice demonstrates the potential of this mucosal vaccine strategy to elicit local and systemic humoral responses.

Heterologous Vaccine Strategy Elicits Robust Neutralizing and Binding Antibody Responses

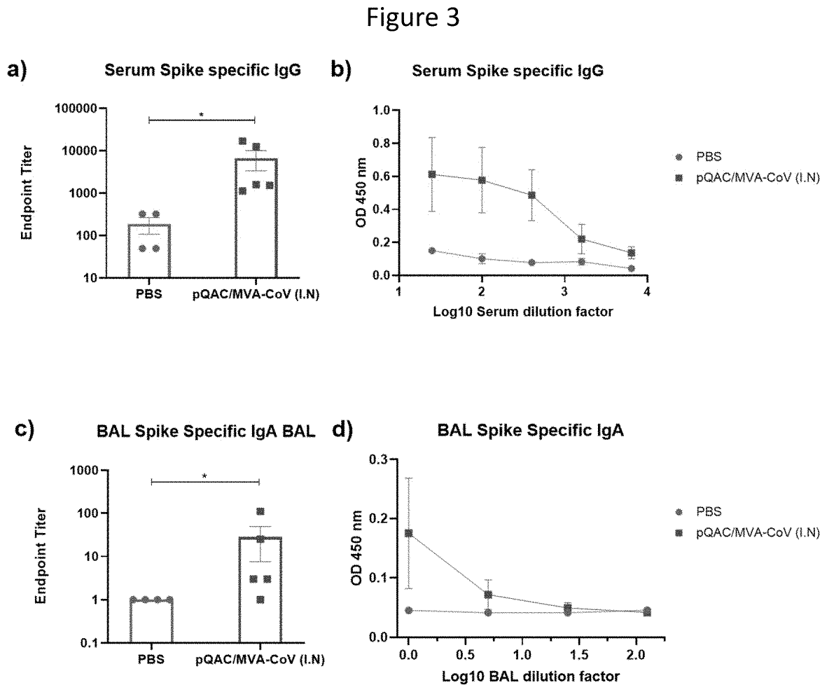

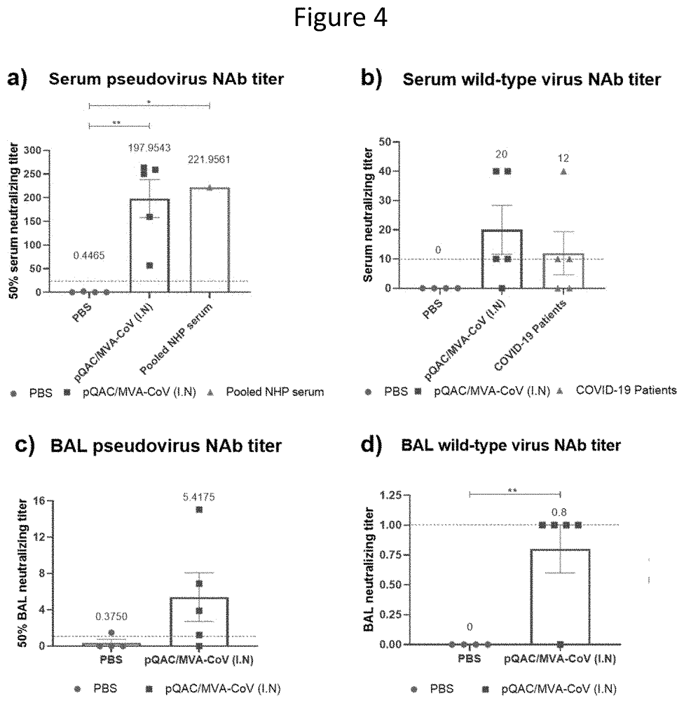

To further characterize and accurately quantify humoral responses in the heterologous vaccine group (pQAC-CoV/MVA-CoV), we evaluated S-specific endpoint titers (EPT) using standard ELISA. Significant antibody titers ranging from about 1:1000-1:15000 for circulating IgG (serum) and 1-1:100 for sIgA (BAL) were detected in the heterologous vaccine group (FIGS. 3a and 3c). To evaluate the neutralizing antibody response, we initially used a pseudovirus neutralization assay developed before using a recombinant lentivirus (25) expressing S protein from SARS-CoV-2. Unlike the PBS group, significant 50% neutralization titers ranging from about 1:50-1:250 (mean: 198) were observed in sera harvested from immunized mice three weeks after the final boost with MVA-CoV (FIG. 4a). The neutralization titers were comparable to the pooled convalescent non-human primate (NHP) serum (BEI Resources-NR-52401) harvested from SARS-CoV-2 infected NHP, demonstrating the ability of the heterologous vaccine strategy to elicit humoral responses to similar levels as seen during SARS-CoV-2 infection. Pseudovirus neutralization titers were also detected in BAL samples of immunized mice, which were higher than those detected in the PBS immunized group (FIG. 4c). Finally, we evaluated the ability of harvested sera and BAL to neutralize wild-type SARS-CoV-2 virus. As observed with the pseudovirus neutralization assay, both serum and BAL samples from vaccinated mice could efficiently neutralize wild-type SARS-CoV-2 (FIGS. 4b and 4d). In fact, the neutralization titers observed with pQAC/MVA-CoV sera were higher than what was observed with COVID-19 patient plasma samples (FIG. 4b). These results highlight the ability of the heterologous vaccine strategy to elicit potent SARS-CoV-2 neutralizing local and systemic antibody responses.

SARS-CoV-2 Specific Cellular Responses in Vaccinated Mice

Next, we evaluated the ability of the experimental vaccines to elicit local and systemic SARS-CoV-2 S- and N-specific cellular immune responses. Intracellular cytokine staining (ICS) was performed with lung cells and splenocytes harvested from vaccinated mice three weeks post final boost (i.e., PB for pQAC-CoV/MVA-CoV and P/2XB for pQAC-CoV alone). For S-specific immune responses, cells were stimulated with purified recombinant spike glycoprotein (BEI resources-NR-52396) from SARS-CoV-2 for 24 h before staining. For N-specific immune responses, cells were stimulated with an N protein-peptide array from the SARS-CoV (BEI Resources-NR-2670) for 5 h before staining based on earlier protocols (26, 27). Increased lung cell death was observed in a few samples, which were excluded from the analysis accordingly.

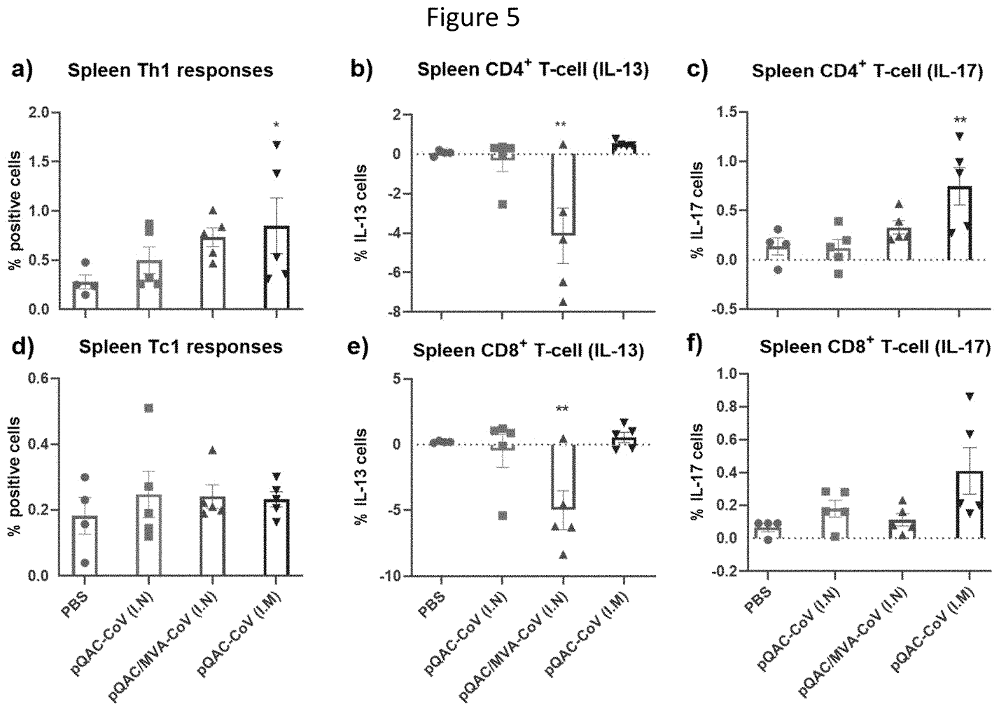

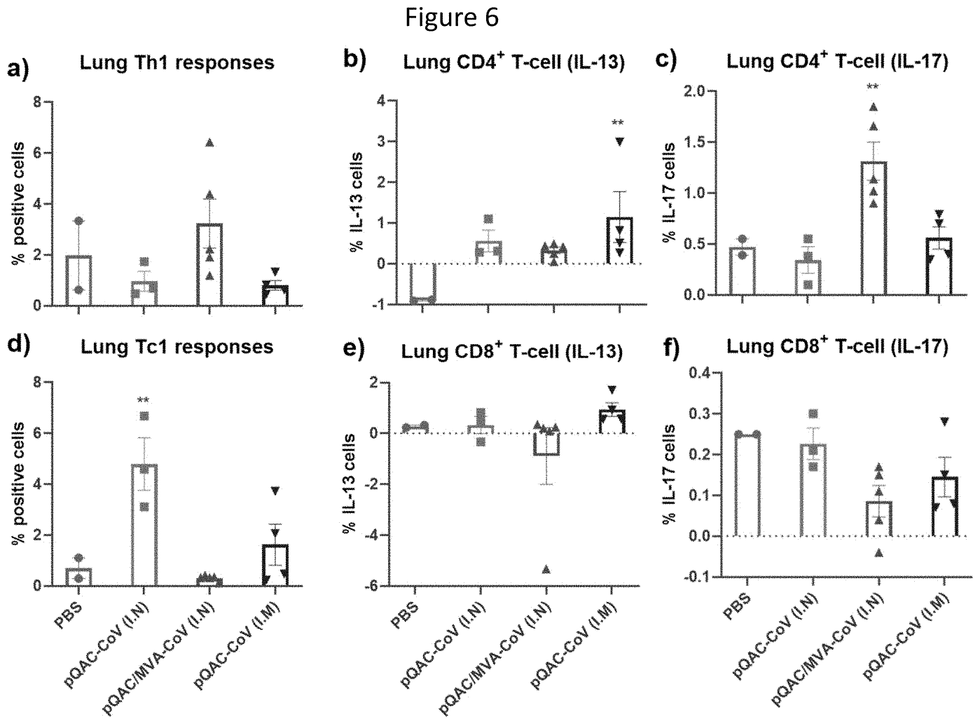

Interestingly, immunization with the heterologous vaccine induced significant S-specific Th17 (IL-17+) responses in the lungs (FIG. 6c). Th2 (IL-13+) and Tc2 responses were significantly lower in the heterologous vaccine group in the spleen and lungs of vaccinated mice (FIGS. 5 and 6). Significantly higher S-specific Tc1 responses (predominantly IFN-γ+) were also present in the lungs of pQAC-CoV (I.N) immunized mice without significant induction of CD4+ T cells (FIG. 10), consistent with observations made following QAC-DNA vaccination in chickens (11). In addition, intramuscular (I.M) administration of pQAC-CoV alone induced significantly higher Th1 and Th17 responses in the spleen of vaccinated mice (FIG. 5). In one group, we sacrificed mice after a single dose administration of pQAC-CoV, which revealed higher induction of TNFα+ and IL2+CD4+ T cells in the lungs (data not shown), emphasizing the ability of pQAC-CoV to elicit early T-cell responses.

Moreover, bias towards Th1 cellular immune responses was observed (IL-2 and TNFα) after prime pQAC-CoV, which was then complemented by a Th17 response after MVA-CoV boost vaccination. T-cell immune responses against N were also evaluated but with no significant induction of CD4+ or CD8+ cells in both lungs and spleen. That being said, a statistically significant reduction in N-specific IL-13 producing CD8 and CD4 T-cells was observed in the lungs and spleen of all experimentally immunized mice (data not shown). These results demonstrate the ability of the heterologous vaccine strategy to elicit both potent local and systemic SARS-CoV-2 neutralizing antibody and cellular immune responses.

SARS-CoV-2 Vaccines Induce Polyfunctional T-Cells

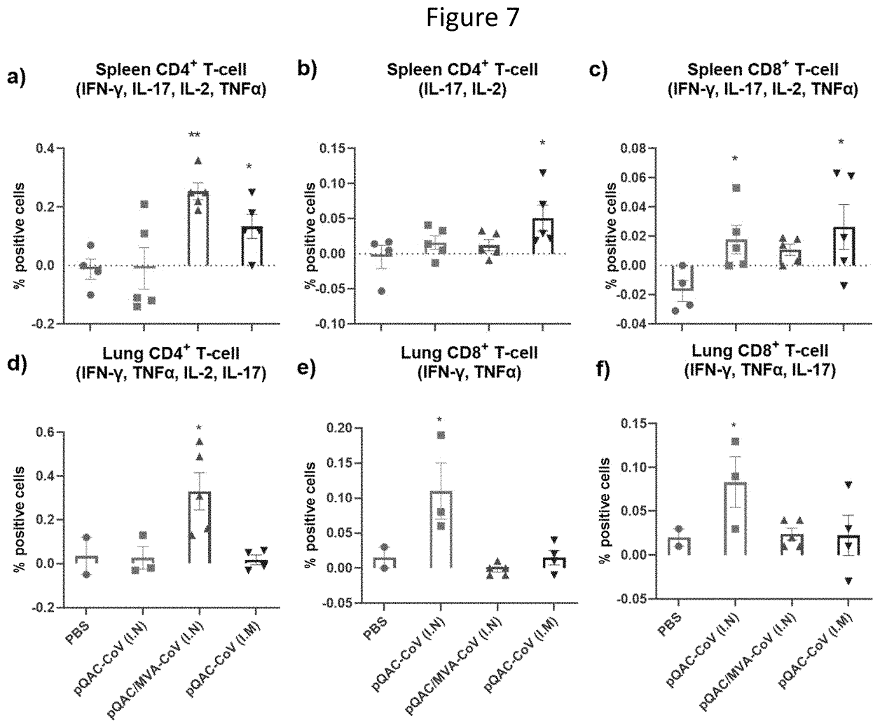

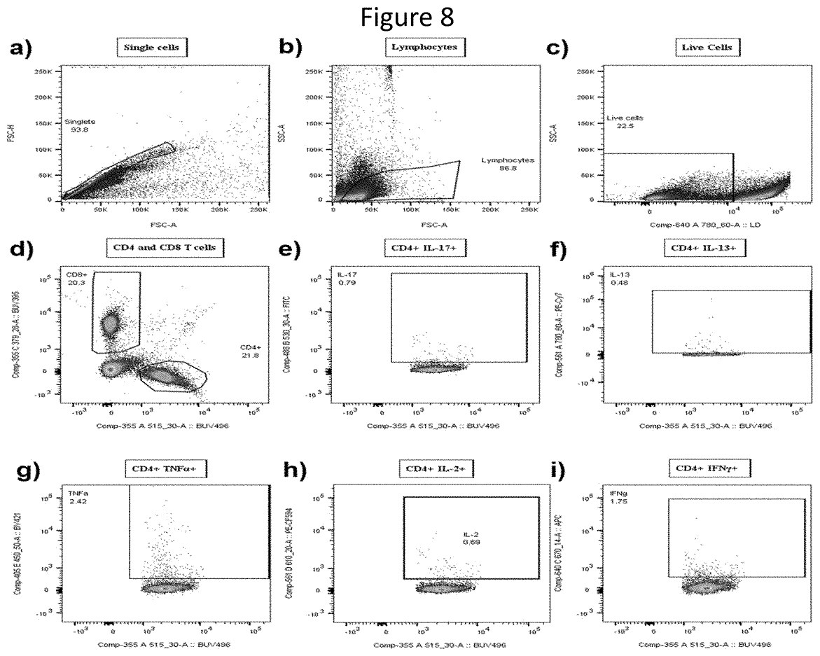

To further characterize cellular immune responses, we investigated the ability of the experimental vaccines to induce polyfunctional T-cells using ICS. Our analysis revealed a significant induction of polyfunctional CD4+ cytokine producing T-cells (IFN-γ, TNFα, IL-2, and 11-17) in the lung and spleen of mice vaccinated with the heterologous vaccine (FIGS. 7a and 7d). In addition, we detected the significant induction of polyfunctional CD4+ T-cells (IL-17, IL-2) and CD8+ T-cells (IFN-γ, TNFα, IL-2+, and 11-17) in the spleens of pQAC-CoV vaccinated mice via the I.M route (FIGS. 7b and 7c). We also detected significant induction of polyfunctional CD8+ T-cells (IFN-γ, TNFα, 11-17 and IFN-γ, TNFα) in the lungs of pQAC-CoV vaccinated mice via the I.N route (FIGS. 7e and 7f). Overall, analysis of cellular immune responses indicated that pQAC-CoV-based vaccines administered via the I.N route induced robust local responses in the lung and those given via I.M route in the spleen, as expected.

### Discussion:

Although many experimental vaccines against SARS-CoV-2 are currently in development and in clinical trials, multiple vaccine approaches will realistically be needed to cover the global population and achieving herd immunity (13-16). As previously established, the upper respiratory mucosa is the primary site of SARS-CoV-2 infection, and local mucosal immune responses may be critical in limiting SARS-CoV-2 dissemination into the lower respiratory tract and subsequent pneumonia (28). Local mucosal immune responses such as airway epithelium T-cell responses and IgA humoral responses are critical for restricting respiratory viral pathogens like SARS, MERS CoV, etc (22, 23, 29, 30). Mucosal vaccination strategies such as intranasal vaccinations could provide an effective therapeutic strategy against COVID-19 by mediating the induction of local and systemic immune responses. Although appealing, formulating intranasal vaccinations can be challenging, owing to the low bioavailability, antigen uptake, and antigen degradation at mucosal surfaces (31, 32). Previously, we detailed the development of the QAC adjuvant system for efficient intranasal delivery of DNA immunogens, circumventing some of the challenges observed with I.N vaccination (11). This rational design specifically targets the development of intranasal vaccine delivery route based on the known mucoadhesive properties of chitosan and the use of the potent immune-stimulant Quil-A for combating mucosal pathogens such as SARS-CoV-2. In this study, we describe the pre-clinical development of a mucosal 2-dose heterologous vaccine candidate against COVID-19 using the QAC adjuvant system. We were able to rapidly develop and characterize plasmid DNA and MVA encoding SARS-CoV-2 spike (S) and nucleocapsid (N) proteins as soon as sequences were available on the GenBank database. PB with pQAC-CoV/MVA-CoV in mice led to the development of neutralizing antibody responses in serum (systemic) and BAL (local). Robust local and systemic T-cell responses against SARS-CoV-2 S protein were also observed in vaccinated mice.

Based upon our previous experience with avian coronavirus and other studies with SARS-CoV-2, we initially chose both nucleocapsid and spike as our antigen targets. Although we immunized mice concurrently with both the N and S vaccine constructs, we focused on characterizing the more biologically relevant humoral responses against spike, which neutralize and limit viral entry (33, 34). Mice immunized with the pQAC/MVA-CoV vaccine candidate induced serum IgG and BAL IgA capable of binding to both full-length spike and the RBD of the spike. Neutralizing antibodies (nAb) against SARS-CoV-2 have been shown to bind to the spike RBD and limit viral entry (34). Likewise, serum and BAL harvested from vaccinated mice were able to efficiently neutralize pseudovirus expressing SARS-CoV-2 spike, which was comparable to neutralization observed with convalescent serum from SARS-CoV-2 infected NHP. Independent studies have shown that convalescing NHP are protected against SARS-CoV-2 and SARS-CoV re-infection, highlighting the potential of neutralizing antibodies induced in pQAC/MVA-CoV immunized mice to prevent SARS-CoV-2 infection (35, 36). Other studies have shown that pseudovirus neutralization correlates positively with infectious virus neutralization and might protect against SARS-CoV-2 challenge (37). Although we did not perform challenge studies, we did observe that sera and BAL harvested from pQAC/MVA-CoV immunized mice were able to neutralize wild-type SARS-CoV-2 efficiently.

Generally, vaccinia viral vectors (e.g., MVA) and chitosan adjuvants induced robust T-cell responses, including induction of pro-inflammatory cytokines like IFN-γ, TNFα, IL-2, 11-17, etc., which have been implicated in limiting a plethora of viral infections such as HSV-1, HSV-2, West Nile virus, SIV, RSV, and Influenza (6, 38-43). Although Tc17 cells, characterized by the secretion of IL-17 themselves, aren't cytotoxic like IFN-g+ CD8+ T-cells, they can directly activate and prime cytotoxic CD8+ T-cells (40). IL-17 ablation in RSV infected mice led to increased airway inflammation and airway mucus, indicating a potential role for IL-17 in limiting inflammation (41). IL-17 is critical for recruiting B cells to the lung in response to influenza infection by inducing CXCL13 expression (42). IL-17 can also promote migration and differentiation of B1a cells, the primary source of IgM production (44).

Interestingly, pQAC/MVA-CoV immunized mice have modest titers of IgA and IgG, and a significant percentage of Th17 cells reactive to SARS-CoV-2 S, which was not observed in other groups. This observation suggests a potential mechanism of action for the heterologous vaccine candidate: that immunization coupled with IL-17 production amplified the SARS-CoV-2 S-specific humoral response. That being said, the presence of IL-17 producing CD4+ T-cells in the spleen of pQAC-CoV (I.M) did not correlate with antibody levels. Concurrent expression of pro-inflammatory cytokines is a hallmark of Tc17 and Th17 cells (45). Similarly, we observed polyfunctional CD8+ T-cells and CD4+ T-cells in the lungs and spleen of pQAC/MVA-CoV immunized mice.

In general, we recognize that the presence of IL-17 among elicited cytokines could be a double-edged sword. For example, increased tissue pathology during viral infections has been observed to be facilitated by promoting a Th2 response, including IL-13 production (45). In our hands, we noticed no change in IL-13 producing cells in the lungs and spleen of pQAC/MVA-CoV vaccinated mice compared to the control group. Previously, Th2 biased responses orchestrated by IL-13 were usually associated with vaccine-associated enhanced respiratory disease (VAERD) (13). T-cell responses elicited against N protein were minimal, which could be explained by the fact that the SARS-CoV N peptide pool was used for stimulation initially when SARS-CoV-2 N peptide array was unavailable. However, reduction in IL-13 production was observed in the vaccinated mice highlighting the absence of Th2 responses.

Interestingly, the pQAC-CoV vaccines administered via the I.N route could induce potent S-specific Tc1, specifically CD8+IFN-γ cells in the lung. Similarly, pQAC-CoV administered via the I.M route was also able to induce potent S specific Th1 and Th17 cellular responses in the spleen. The roles of local and systemic IFN-γ producing T-cells in limiting coronaviruses and other respiratory viral pathogens have been reported previously shown in multiple reports (46).

Overall, the work presented here provided a proof of concept that a PB strategy with a heterologous vector system (in this case, pQAC/MVA-CoV) can elicit robust humoral and cellular responses against SARS-CoV-2. This strategy enjoyed the advantages of mucosal immunization detailed before and could be an example of developing rapid vaccines against COVID-19 and other future respiratory infections. Nonetheless, the protective efficacy and full safety profile of the described vaccines are yet to be evaluated. Specifically, the immune interference effects of the N antigen, if any, need further exploration. However, preliminary studies suggested no dampening of S-specific immune responses when N antigen was used in our vaccine. A similar outcome could be envisioned for the pQAC/MVA-CoV vaccines.

### REFERENCES

1. F. Zhou et al., Clinical course and risk factors for mortality of adult inpatients with COVID-19 in Wuhan, China: a retrospective cohort study. The Lancet 395, 1054-1062 (2020).
2. N. Zhang, S. Jiang, L. Du, Current advancements and potential strategies in the development of MERS-CoV vaccines. Expert Rev Vaccines 13, 761-774 (2014).
3. F. Luo et al., Evaluation of Antibody-Dependent Enhancement of SARS-CoV Infection in Rhesus Macaques Immunized with an Inactivated SARS-CoV Vaccine. Virol Sin 33, 201-204 (2018).
4. N. Wang, J. Shang, S. Jiang, L. Du, Subunit Vaccines Against Emerging Pathogenic Human Coronaviruses. Front Microbiol 11, 298 (2020).
5. B. S. Graham, J. R. Mascola, A. S. Fauci, Novel Vaccine Technologies: Essential Components of an Adequate Response to Emerging Viral Diseases. JAMA 319, 1431-1432 (2018).
6. A. F. Altenburg et al., Modified Vaccinia Virus Ankara (MVA) as Production Platform for Vaccines against Influenza and Other Viral Respiratory Diseases. Viruses Basel 6, 2735-2761 (2014).
7. C. I. Paules, H. D. Marston, A. S. Fauci, Coronavirus Infections-More Than Just the Common Cold. JAMA, (2020).
8. A. M. Talaat, R. Lyons, S. A. Johnston, A combination vaccine confers full protection against co-infections with influenza, herpes simplex and respiratory syncytial viruses. Vaccine. 20, 538-544 (2001).
9. P. Cai, X. Zhang, M. Wang, Y. L. Wu, X. Chen, Combinatorial Nano-Bio Interfaces. ACS Nano 12, 5078-5084 (2018).
10. D. H. a. M. Bros, DNA Vaccines—How Far From Clinical Use? Int J Mot Sci. 19, (2018).
11. S. S. Chandrasekar, B. A. Kingstad-Bakke, C. W. Wu, M. Suresh, A. M. Talaat, A Novel Mucosal Adjuvant System for the Immunization Against Avian Coronavirus Causing Infectious Bronchitis. J Virol, (2020).
12. N. v. D. Emmie de Wit, Darryl Falzarano and Vincent J. Munster, SARS and MERS: recent insights into emerging coronaviruses. Nature Reviews Microbiology 14, (2016).
13. K. S. Corbett et al., Evaluation of the mRNA-1273 Vaccine against SARS-CoV-2 in Nonhuman Primates. N Engl J Med, (2020).
14. N. van Doremalen et al., ChAdOxl nCoV-19 vaccination prevents SARS-CoV-2 pneumonia in rhesus macaques. bioRxiv, 2020.2005.2013.093195 (2020).
15. T. R. F. Smith et al., Immunogenicity of a DNA vaccine candidate for COVID-19. Nature Communications 11, (2020).
16. P. F. McKay et al., Self-amplifying RNA SARS-CoV-2 lipid nanoparticle vaccine candidate induces high neutralizing antibody titers in mice. Nat Commun 11, 3523 (2020).
17. F. Tang et al., Lack of Peripheral Memory B Cell Responses in Recovered Patients with Severe Acute Respiratory Syndrome: A Six-Year Follow-Up Study. Journal of Immunology 186, 7264-7268 (2011).
18. H. Peng et al., Long-lived memory T lymphocyte responses against SARS coronavirus nucleocapsid protein in SARS-recovered patients. Virology 351, 466-475 (2006).
19. J. Seow et al., Longitudinal evaluation and decline of antibody responses in SARS-CoV-2 infection. medRxiv, 2020.2007.2009.20148429 (2020).
20. C. Maeto, A. M. Rodriguez, M. P. Holgado, J. Falivene, M. M. Gherardi, Novel mucosal DNA-MVA HIV vaccination in which DNA-IL-12 plus cholera toxin B subunit (CTB) cooperates to enhance cellular systemic and mucosal genital tract immunity. PLoS One 9, e107524 (2014).
21. M. Manrique et al., Nasal DNA-MVA SIV vaccination provides more significant protection from progression to AIDS than a similar intramuscular vaccination. Mucosal Immunol 2, 536-550 (2009).
22. Y. X. Chao, O. Rotzschke, E. K. Tan, The role of IgA in COVID-19. Brain Behav Immun 87, 182-183 (2020).
23. D. Sterlin et al., IgA dominates the early neutralizing antibody response to SARS-CoV-2. medRxiv, 2020.2006.2010.20126532 (2020).
24. B. Corthesy, Multi-faceted functions of secretory IgA at mucosal surfaces. Frontiers in Immunology 4, (2013).
25. K. H. D. Crawford et al., Protocol and Reagents for Pseudotyping Lentiviral Particles with SARS-CoV-2 Spike Protein for Neutralization Assays. Viruses 12, (2020).
26. A. Kamlangdee, B. Kingstad-Bakke, J. E. Osorio, Mosaic H5 Hemagglutinin Provides Broad Humoral and Cellular Immune Responses against Influenza Viruses. Journal of Virology 90, 6771-6783 (2016).
27. A. Kamlangdee, B. Kingstad-Bakke, T. K. Anderson, T. L. Goldberg, J. E. Osorio, Broad Protection against Avian Influenza Virus by Using a Modified Vaccinia Ankara Virus

Expressing a Mosaic Hemagglutinin Gene. Journal of Virology 88, 13300-13309 (2014).
28. R. Wolfel et al., Virological assessment of hospitalized patients with COVID-2019. Nature 581, 465-469 (2020).
29. A. Mubarak, W. Alturaiki, M. G. Hemida, Middle East Respiratory Syndrome Coronavirus (MERS-CoV): Infection, Immunological Response, and Vaccine Development. J Immunol Res 2019, U.S. Pat. No. 6,491,738 (2019).
30. P. R. Hsueh, L. M. Huang, P. J. Chen, C. L. Kao, P. C. Yang, Chronological evolution of IgM, IgA, IgG and neutralisation antibodies after infection with SARS-associated coronavirus. Clin Microbiol Infect 10, 1062-1066 (2004).
31. O. Borges, G. Borchard, J. C. Verhoef, A. de Sousa, H. E. Junginger, Preparation of coated nanoparticles for a new mucosal vaccine delivery system. Int J Pharm 299, 155-166 (2005).
32. B. A. Kingstad-Bakke et al., Effective mosaic-based nanovaccines against avian influenza in poultry. Vaccine 37, 5051-5058 (2019).
33. J. Prevost et al., Cross-sectional evaluation of humoral responses against SARS-CoV-2 Spike. bioRxiv, 2020.2006.2008.140244 (2020).
34. G. Siracusano, C. Pastori, L. Lopalco, Humoral Immune Responses in COVID-19 Patients: A Window on the State of the Art. Front Immunol 11, 1049 (2020).
35. C. T. Tseng et al., Immunization with SARS coronavirus vaccines leads to pulmonary immunopathology on challenge with the SARS virus. PLoS One 7, e35421 (2012).
36. A. Chandrashekar et al., SARS-CoV-2 infection protects against rechallenge in rhesus macaques. Science 369, 812-817 (2020).
37. J. Yu et al., DNA vaccine protection against SARS-CoV-2 in rhesus macaques. Science 369, 806-811 (2020).
38. P. Bagri et al., Novel Role for Interleukin-17 in Enhancing Type 1 Helper T Cell Immunity in the Female Genital Tract following Mucosal Herpes Simplex Virus 2 Vaccination. J Virol 91, (2017).
39. V. C. Anipindi et al., Estradiol Enhances CD4+ T-Cell Anti-Viral Immunity by Priming Vaginal DCs to Induce Th17 Responses via an IL-1-Dependent Pathway. PLoS Pathog 12, e1005589 (2016).
40. D. Acharya et al., Interleukin-17A Promotes CD8+ T Cell Cytotoxicity To Facilitate West Nile Virus Clearance. J Virol 91, (2017).
41. H. Huang, J. Saravia, D. You, A. J. Shaw, S. A. Cormier, Impaired gamma delta T cell-derived IL-17A and inflammasome activation during early respiratory syncytial virus infection in infants. Immunol Cell Biol 93, 126-135 (2015).
42. X. Wang et al., A critical role of IL-17 in modulating the B-cell response during H5N1 influenza virus infection. Cell Mol Immunol 8, 462-468 (2011).
43. N. Riteau, A. Sher, Chitosan: An Adjuvant with an Unanticipated STING. Immunity 44, 522-524 (2016).
44. X. Wang et al., IL-17A Promotes Pulmonary B-1a Cell Differentiation via Induction of Blimp-1 Expression during Influenza Virus Infection. PLoS Pathog 12, e1005367 (2016).
45. W. T. Ma, X. T. Yao, Q. Peng, D. K. Chen, The protective and pathogenic roles of IL-17 in viral infections: friend or foe? Open Biol 9, 190109 (2019).
46. F. Novelli, J. L. Casanova, The role of IL-12, IL-23 and IFN-gamma in immunity to viruses. Cytokine Growth Factor Rev 15, 367-377 (2004).
47. R. Hernandez, & Brown, D. T., Growth and Maintenance of Chick Embryo Fibroblasts (CEF). (John Wiley & Sons, Inc, Current Protocols in Microbiology, May 2010).
48. B. R. Stading et al., Infectivity of attenuated poxvirus vaccine vectors and immunogenicity of a raccoonpox vectored rabies vaccine in the Brazilian Free-tailed bat (Tadarida brasiliensis). Vaccine 34, 5352-5358 (2016).
49. J. Valosky, H. Hishiki, T. E. Zaoutis, S. E. Coffin, Induction of mucosal B-cell memory by intranasal immunization of mice with respiratory syncytial virus. Clin Diagn Lab Immunol 12, 171-179 (2005).

### Example 2. Systemic Responses More than Local Immune Responses Induced by Nanoparticle Vaccines are Critical for the Control of SARS-CoV-2 in Humanized Mice Model

### Results

Nanoparticles Formed by QAC Encapsulation of Plasmid DNA Immunogens

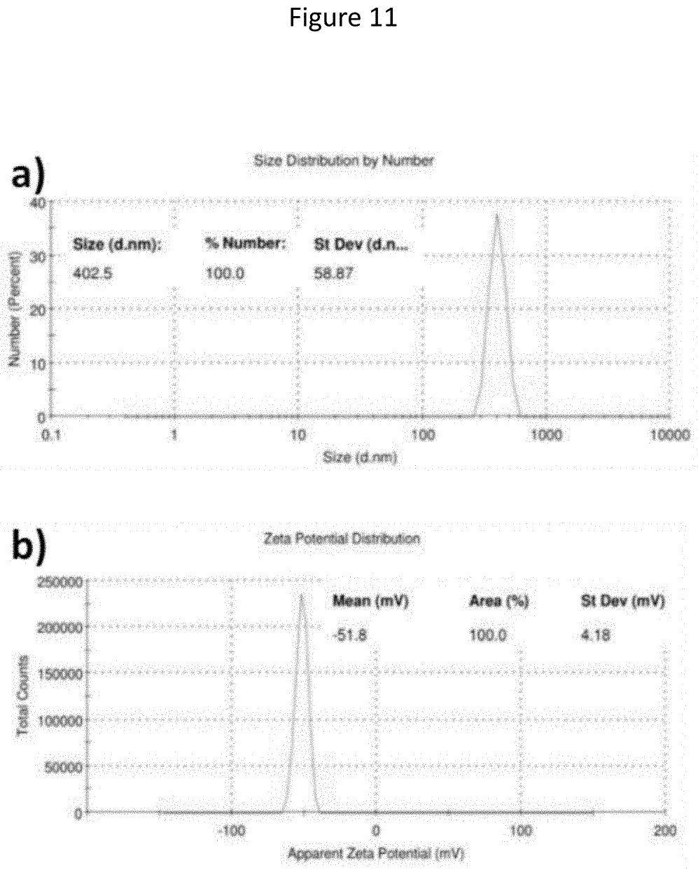

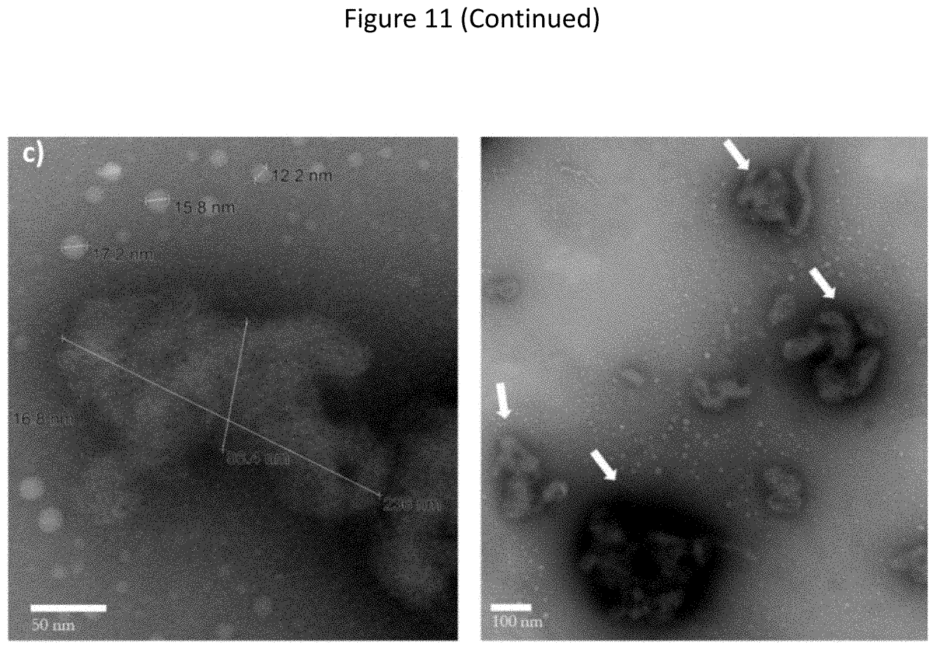

Uptake and subsequent cellular immune responses generated is influenced by the size of the nanoparticle (NP) vaccines. Plasmid DNA vector pCAGGS encoding for the SARS-CoV-2, Wuhan-Hu-1 (GenBank: MN908947) Spike glycoprotein (pCoV-S) procured through BEI resources was used for size and charge characterization (NR-52394). QAC complexation of pCoV-S suitable for mice inoculation led (pQAC-S) to formation of nanoparticles around 400 nm using dynamic light scattering (DLS) analysis post sonication for disaggregation with a net positive zeta potential of −51.9±4.58 mV (FIG. 11b). As expected, transmission electron microscopy (TEM) scanning revealed the presence of QAC-plasmid DNA complexes >200 nm in size (FIG. 11c). QAC adjuvant system is non-toxic, mediates sustained and stable release of packaged DNA

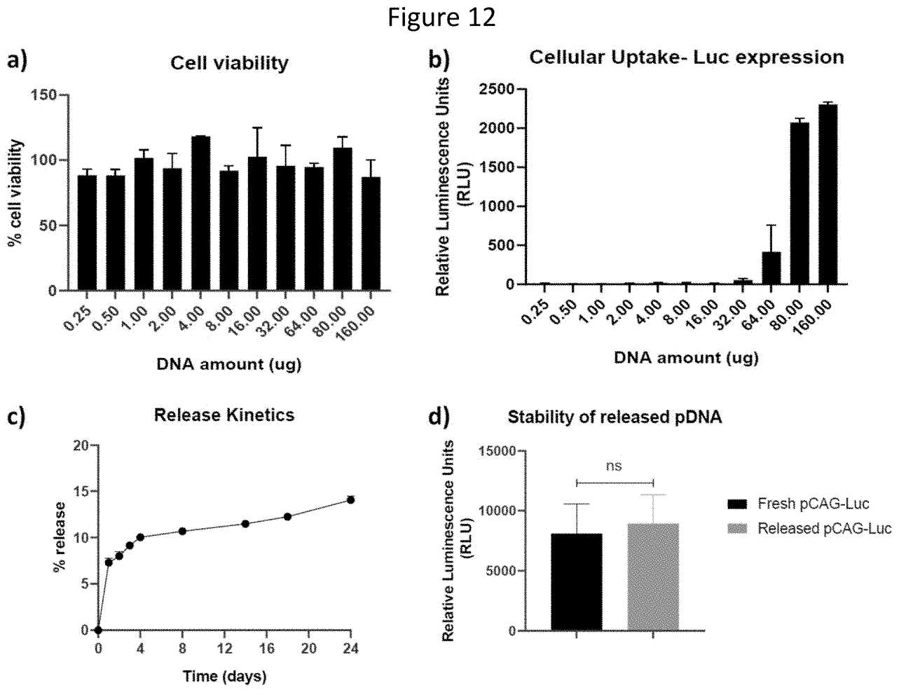

We have previously shown that QAC based particles are well tolerated in poultry hosts. To evaluate the biocompatibility of QAC based vaccines in a relevant cell culture model for SARS-CoV-2 vaccines, different amounts of QAC encapsulated pCAG vector encoding the Luciferase gene (pQAC-Luc) was added to HEK 293T cells and cell viability after 3 days measured using MTT assay. Interestingly, no cytotoxicity were observed in cells post pQAC-Luc addition even at high DNA amounts highlighting the safety of QAC based vaccines (FIG. 12a). The delivery of plasmid DNA by QAC NPs was qualitatively determined previously using GFP reporter plasmid. To quantitatively determine delivery of cargo plasmid, pQAC-Luc was added to HEK 293T cells in increasing amounts and luciferase expression assayed 3 days post addition. As expected, luciferase expression was detected in a dose dependent manner, indicating delivery and expression of luciferase from the packaged construct (FIG. 12b). The release kinetics of plasmid DNA from pQAC-Luc was evaluated in phosphate buffered saline (PBS) at physiological temperature (37° C.) and pH (7.4) by quantifying the amount of DNA released over time using spectrophotometry. Close to 15% of the packaged pCAG-Luc was released over 24 days in a sustained manner (FIG. 12c). To further confirm that the released DNA was functional, pCAG-Luc released 24 hrs after incubation was used to transfect HEK 293T cells with standard transfection reagent. No difference in luciferase expression was observed between control fresh pCAG-Luc and released pCAG-Luc underscoring the ability of the QAC adjuvant system to release functional and stable plasmid DNA (FIG. 12d).

Internalization of Plasmid DNA by Macrophages when Complexed with QAC Adjuvant System

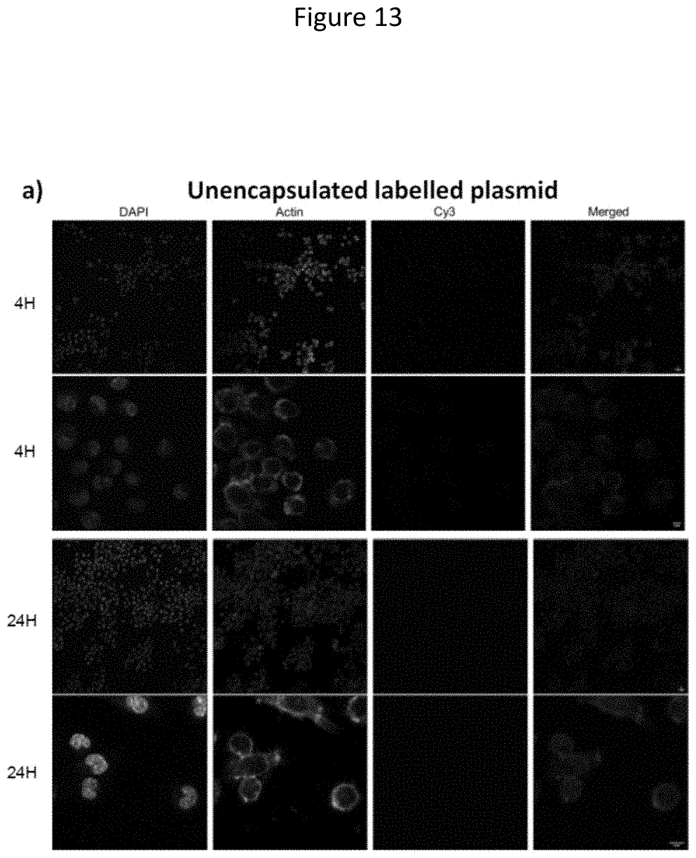

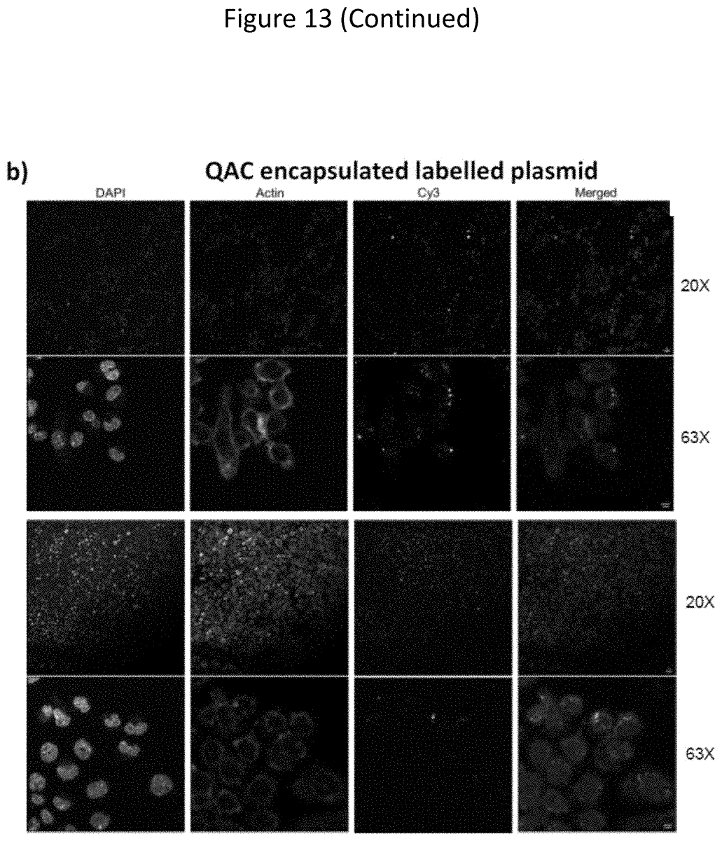

Upon immunization via the parenteral route, for optimal efficacy, vaccine antigens need to be taken up by resident immune cells, like dendritic cells and macrophages. J774 murine macrophages were used to evaluate the ability of QAC to mediate delivery of plasmid DNA to target immune cells. J774 murine cells were incubated with fluorescently labeled plasmid DNA encapsulated by QAC (pQAC-Cy3) and fluorescent microscopy used to determine uptake at two different time points (4 and 24 hrs). These times were chosen to monitor uptake of plasmid DNA over time. Cy3 labelled plasmid DNA was taken up efficiently when delivered by QAC (FIG. 13b) which was not observed with unencapsulated labeled plasmid (FIG. 13a) underscoring the need for QAC complexation. Internalization was observed as early as 4 hrs post addition and maintained for 24 h post addition (FIG. 13b). Qualitatively more labelled plasmid DNA was observed at the 4 hrs time point than 24 hrs. Labelled plasmid DNA was also observed in the nucleus of J774 cells (DAPI stained) indicating favorable localization to nucleus which should promote expression of vaccine antigens encoded by the plasmid DNA as confirmed by our luciferase expression studies above (FIG. 12b). Overall, our analysis indicates that the QAC adjuvant system is well tolerated in cell culture and can mediate delivery of plasmid DNA to target immune cells.

Systemic SARS-CoV-2 Specific Immune Responses in Vaccinated Mice

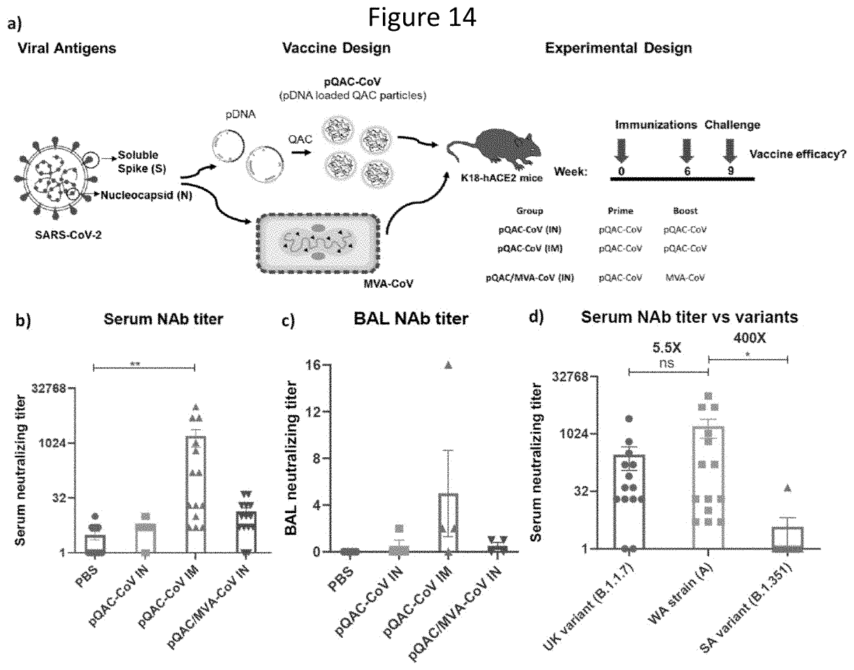

QAC complexed plasmid DNA vaccine encoding for SARS-CoV-2 S and N has been shown to be immunogenic when administered via multiple routes (intramuscular, IM and intranasal, IN) and either via homologous (DNA priming followed by DNA boosting) or heterologous vaccination (DNA priming followed by MVA vaccination encoding the same antigens) in C57BL/6 mice. Although immunogenic, each vaccination strategy had a different immune profile with parenteral vaccination leading to systemic immune responses and IN vaccination leading to local immune responses. To investigate which of these immune responses are truly protective against SARS-CoV-2 challenge, K18-hACE2 mice were utilized in this study. Groups of K18-hACE2 mice were immunized with QAC complexed plasmid DNA (pCoV-S and pCoV-N, both termed pQAC-CoV) via IN or IM routes followed by boosting 6 weeks post initial immunization (FIG. 14a). Another group of K18-hACE2 mice was also primed with pQAC-CoV, followed by a boosting with MVA-CoV (MVA-S and MVA-N) via the IN route 6 weeks after prime (FIG. 14a). pQAC-CoV parenteral administration led to the significant induction of SARS-CoV-2 neutralizing antibody titers (nAb) in harvested sera 3 weeks post final vaccination (wpv, FIG. 14b). nAb levels were also higher in the pQAC/MVA-CoV IN administration as reported previously, albeit non-significant (FIG. 14b). Interestingly, BAL harvested from pQAC-CoV IM vaccinated mice was able to neutralize SARS-CoV-2 at levels higher than detected with the unvaccinated group (FIG. 14c). Next, we investigated the ability of sera from pQAC-CoV IM vaccinated mice to neutralize common circulating variants of SARS-CoV-2, B.1.17 and B.1.351. Neutralization titers against B.1.17 showed no significant difference when compared to SARS-CoV-2 strain A against which the vaccines are developed (FIG. 14d). In contrast, neutralization titers against B.1.351 were non-detectable and significantly lower, an observation also seen with other approved COVID-19 vaccines (FIG. 14d). Our results suggest that parenteral QAC based immunization is better at inducing significant systemic neutralizing immune responses than local immunization.

Mucosal Administration of SARS-CoV-2 Vaccine Induces Local Cellular Responses

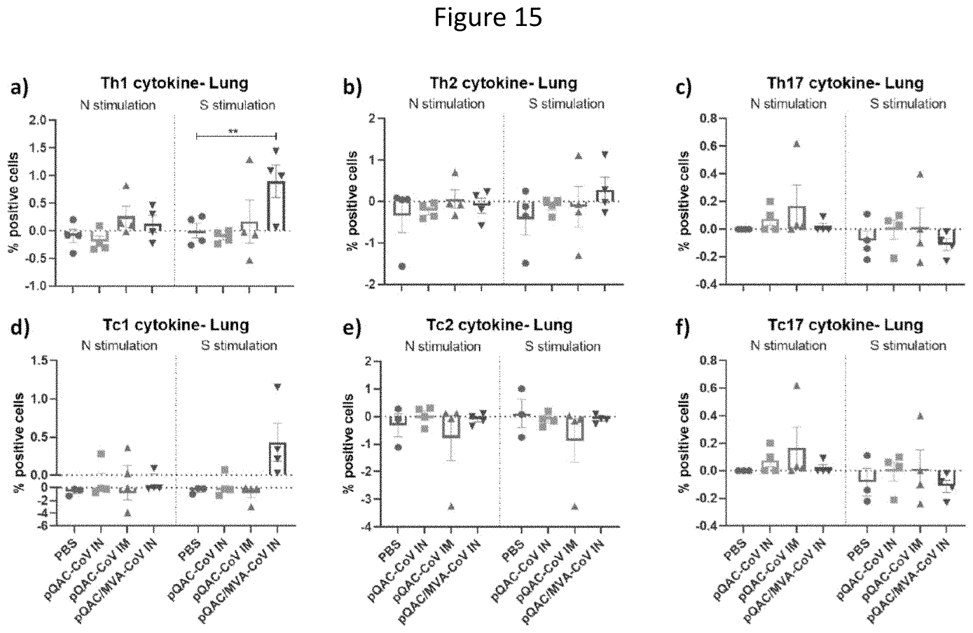

Previously we have shown that QAC based SARS-CoV-2 vaccines elicited significant local (lungs) and systemic (spleen) type-1 T-cell responses. Intracellular cytokine staining (ICS) was performed with lung cells harvested from vaccinated mice three weeks post final boost (pre-challenge) to evaluate local immune responses elicited by QAC based vaccines. For S and N-specific immune responses, cells were stimulated with purified recombinant spike glycoprotein (BEI resources-NR-52396) and N Protein N-terminal RNA binding domain from SARS-CoV-2 (BEI resources-NR-53246) respectively overnight before staining. As seen previously, pQAC/MVA-CoV vaccination led to the significant induction of type 1 helper (Th1, FIG. 15a) and cytotoxic (Tc1, FIG. 15d) T-cell responses (IFN-γ or TNFα or IL-2+) post SARS-CoV-2 S stimulation. Detectable immune responses were not elicited by the 2-dose IN administration of pQAC-CoV potentially highlighting the beneficial impact of mixing and matching vaccination platforms as seen with pQAC-MVA-CoV (FIGS. 15a-f). As expected, no induction of deleterious type-2 (IL-4+ or IL-13+) immune responses was observed in the lungs from any of the vaccinated groups (FIGS. 15b, e). Interestingly, detectable cytokine producing T-cells were only observed with S stimulation and not with N stimulation in any of the vaccinated mice (FIGS. 15a-d). Also, pQAC-CoV IM administration did not lead to the induction of T-cell immune responses in the lungs (FIGS. 15a-f). These results indicate that pQAC/MVA-CoV IN administration elicits better local lung immune T-cell responses in contrast to pQAC-CoV IM administration that elicits better systemic antibody responses.

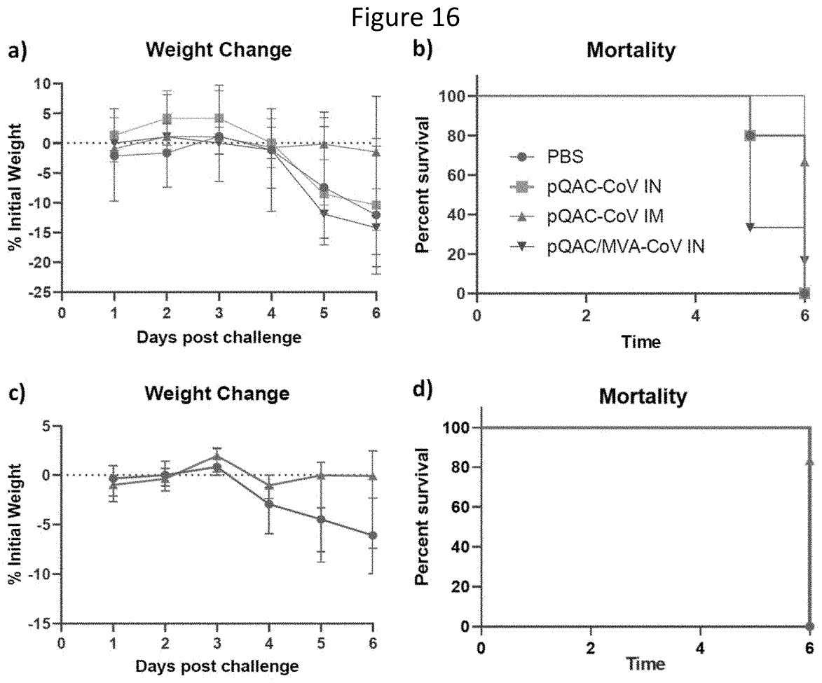

A follow-up study was conducted with only the PBS and pQAC-CoV IM vaccinations for reproducibility and to validate the findings of the first trial. The output from the follow-up trial are depicted in fig c and d of Fig panels 16-19. Mice were monitored for weight loss, survivability, infectious viral load in the lungs and brain and pathology in lungs, spleen and brains of the vaccinated mice. Unvaccinated mice started losing weight at 4 days post challenge (dpc) with all mice meeting criteria for euthanasia by 6 dpc (FIGS. 16a, c). In contrast, a majority of the pQAC-CoV IM administered mice (65-80%) survived and showed no apparent clinical signs of SARS-CoV-2 infection with no weight loss (FIGS. 16a, c). pQAC/MVA-CoV IN offered minimal protection with significant weight loss observed comparable to the PBS group and only 20% of the mice surviving at experimental end point (FIGS. 16a-d). Mice vaccinated with pQAC-CoV IN offered no protection with all the mice following a similar clinical trajectory to the unvaccinated mice (FIGS. 16b, d). Parenteral administration of pQAC-CoV which induces significant systemic immune responses provided better protection when compared to both the mucosal vaccine administrations.

Reduced Viral Burden in the Lungs and Brains of Vaccinated Humanized Mice

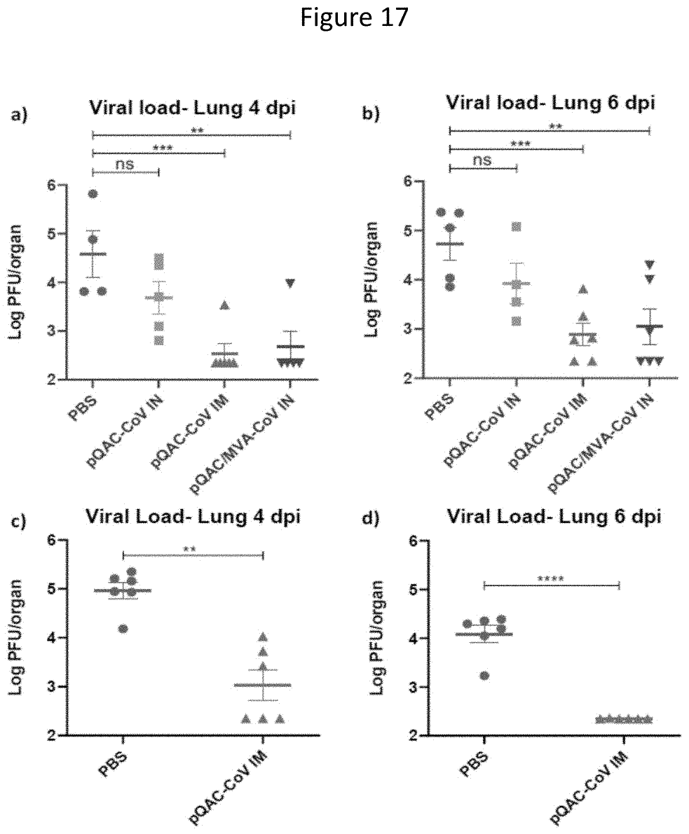

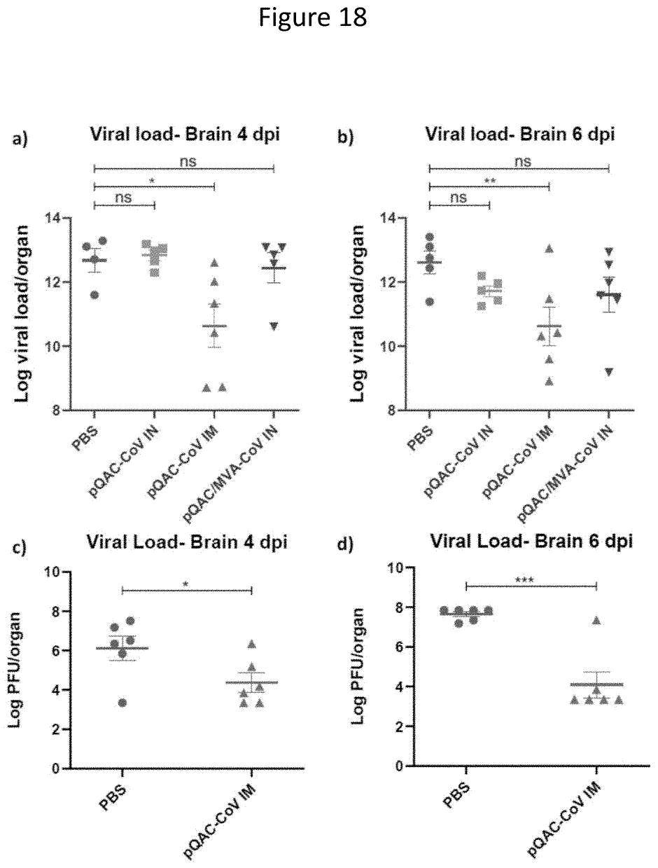

We quantified the infectious viral load in the lungs and brains of challenged mice at two different time points, 4 and 6 dpc. We noticed a significant reduction in the lung viral load of both pQAC-CoV IM and pQAC/MVA-CoV IN vaccinated mice when compared to the unvaccinated mice across both time points (FIG. 17a-d). In contrast, only pQAC-CoV IM vaccinated mice had significantly lower viral RNA and infectious viral loads in the brain (FIGS. 18a-d). In agreement with the immunology findings, we did not see any significant reduction in the viral loads of pQAC-CoV IN administered mice both in the lungs and brain (FIGS. 17a-d, 18a-d). Overall, the viral load data correlates well with the clinical findings with pQAC-CoV IM administration offering the best protection reducing local viral replication in the lungs and limiting viral dissemination to the brains. Interestingly, pQAC/MVA-CoV IN which induces local T-cell responses in the lungs limits viral replication in the lungs but is unable to prevent dissemination to the brain underscoring the need for systemic immune responses for optimal protection in K18-hACE2 mice.

Reduced Viral Pneumonia and Tissue Damage in the Lungs, Spleen and Brains of Vaccinated Humanized Mice

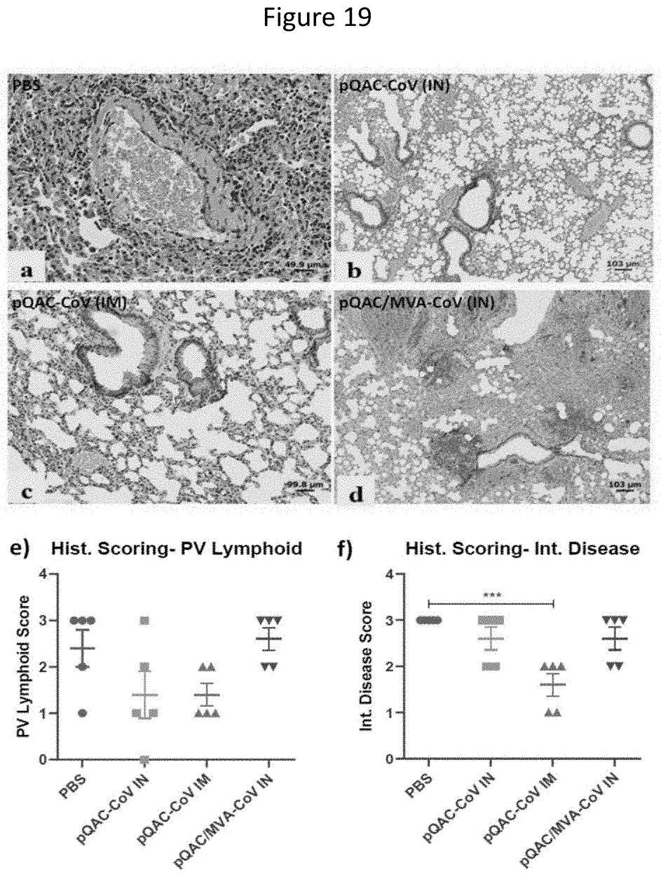

To further examine the efficacy of the different experimental vaccines, hematoxylin and eosin (H&E) staining of lung, spleen and brain sections from vaccinated mice at 6 dpc was conducted and histopathological changes examined in a blinded fashion. Examinations of lungs from unvaccinated mice showed 75% of lung reaction including massive diffuse blood vessels congestion with robust aggregates forming circumferential perivascular lymphoid cuffs and compression of adjacent parenchyma. Furthermore, severe interstitial pneumonia appeared marked as mononuclear cell infiltrates in the interstitial tissue septa and lumen with consolidation and examination of lungs from pQAC-CoV IN vaccinated mice revealed 25% of lung reaction in the form of multi-focal blood vessels congestion together with moderate degree of interstitial pneumonia. In contrast, pQAC-CoV IM vaccinated group reported very mild lungs lesions in comparison with the unvaccinated mice and with only 10% of lung reaction in the form of focal blood vessels congestion with very few perivascular solitaries lymphoid cells aggregates and mild interstitial pneumonia (FIG. 19c). Examination of lungs from pQAC/MVA-CoV IN vaccinated group denoted 50% of lung reaction in the form of diffuse blood vessels congestion with perivascular lymphoid cuffing, together with less severe interstitial pneumonia than the unvaccinated group (FIG. 19d). To quantify the difference in severity, the interstitial disease in each lung sample (H score) was scored taking into account alveolar septal infiltration, extension into the airspaces, and associated edema and atelectasis. As shown in FIG. 19f, pQAC-CoV IM vaccinated mice showed the least sign of interstitial disease at 6 dpc. Mononuclear perivascular lymphoid (PV) infiltration immediately post-challenge can be favorable indicative of optimal vaccination, although no significant difference in the PV scoring was observed between vaccinated and control groups (FIG. 19e). Similar pathology results were noted in the other organs of pQAC-CoV IM vaccinated mice with very few splenic and brain lesions and minimum tissue damage in comparison to the unvaccinated mice which correlates well with the clinical outcome and viral load data. Overall, our analysis indicates that systemic immunity in the form of significant nAb responses elicited by pQAC-CoV IM are critical for protection against SARS-CoV-2 and that local T-cell immune responses such as those elicited by pQAC/MVA-CoV might be sufficient only for controlling viral replication in the lungs but cannot prevent viral dissemination.

### Materials and Methods

Cells and Viruses HEK 293T cells and Vero E6 cells were a kind gift from Dr. Jorge Osorio and were maintained in DMEM supplemented with 10% fetal bovine serum (FBS) and penicillin-streptomycin (D10) at 37° C., 5% CO2 atmosphere. J774 cells were maintained in RPMI supplemented with 10% fetal bovine serum (FBS) and penicillin-streptomycin (D10) at 37° C., 5% CO2 atmosphere.

Preparation of SARS-CoV-2 Vaccine Constructs

QAC and MVA based SARS-CoV-2 vaccine constructs were developed as described previously. Sequences for the SARS-CoV-2 Nucleocapsid (N) were downloaded (GenBank accession number MN908947), back-translated, and codon-optimized for expression in mice. Vector pCAGGS Containing the SARS-Related Coronavirus 2, Wuhan-Hu-1 Spike Glycoprotein Gene (soluble, stabilized), NR-52394 was obtained through BEI Resources, NIAID, NIH. To confirm the insertion of genes in the correct orientation, DNA sequencing was performed at the UW-Madison Biotechnology Center with an ABI Prism 3730XL DNA analyzer using Big Dye terminators (Applied Biosystems, CA). The MVA expressing N and TrS constructs were generated as described before in CEF cells[23]. Plasmid loaded QAC particles were synthesized as described previously [11].

Characterization of Nanoparticles

Size distribution and zeta potential of QAC-NPs in aqueous dispersion was measured by dynamic light scattering (DLS) on a Malvern zeta sizer instrument at 25° C. For size distribution, 10 ul of QAC-NPs in solution was diluted to 3 ml using nuclease free water and placed in a low volume cuvette and analyzed directly. For zeta potential measurement, approximately 1 mL of the diluted QAC-NPs in solution was placed in a disposable capillary zeta potential cell available from the Zeta sizer Nano series. TEM experiments were performed at the Medical school Electron Microscopy facility of the University of Wisconsin-Madison using a Philips CM120 transmission electron microscope (FEI, Eindhoven, the Netherlands) at 80 kV. The size and morphology of vaccine preparations was reexamined via negative staining using the drop method. For the release kinetics assay, QAC NPs loaded with 50 ug total DNA was resuspended in 50 μL of 0.05 M phosphate buffered saline (PBS, pH 7.4) at 37° C. in duplicates. At each time point, suspensions were removed and centrifuged at 14,000 rcf for 20 min. The supernatant was removed and replaced with PBS and returned to incubation. Supernatant samples were quantified for released DNA from the QAC using a GE Healthcare/Amersham Biosciences Ultrospec 3100 ProUV/Visible Spectrophotometer and compared to the total DNA used. For the delivery experiment, pQAC-Luc was added to HEK 293T cells in 96-well plate format at different DNA amounts listed. 72 hours post addition, cells were lysed, and luciferase activity was measured using the ONE-Glo™ Luciferase Assay System (Promega, WI, USA). RLU were measured using the TD 20/20 Lumi-nometer (Turner Designs, CA, USA). For stability studies, HEK 293T cells seeded in 96-well format was transfected with fresh or released pCAG-Luc in the supernatant (24 hrs) from the release kinetics assay using TransTT®-293 Transfection Reagent according to manufacturer's instructions (Minis Bio, WI, USA). Three days post-transfection, cells were lysed, and luciferase activity was measured using the ONE-Glo™ Luciferase Assay System (Promega, WI, USA). RLU were measured using the TD 20/20 Lumi-nometer (Turner Designs, CA, USA).

J774 Uptake Experiment

To evaluate the internalization of QAC-NPs, J774 cells (Mus musculus macrophage cells) were plated in at density of 0.5×106 cells/mL in a 24-well plate with coverslips and incubated overnight at 37° C. The following day, cells were incubated with Cy3 labeled plasmid DNA (LabelIT® Plasmid Delivery Controls, MIR7904) encapsulated by QAC NPs (2 ug/well) for 4h or 24 h. Cells were then washed with phosphate buffered saline (PBS, pH 7.4) to remove non-adherent or loosely adherent NPs and fixed in 4% paraformaldehyde (methanol free). Cells were permeabilized with 0.1% Triton X-100 in PBS for 3 min and washed with PBS. Actin staining was performed by incubating cells with Alexa Fluor 647 Phalloidin (Life Technologies, NY) for 20 min in PBS at room temperature. Coverslips containing stained cells were washed and mounted on glass slides using ProLong with DAPI (Life Technologies, NY). Confocal microscopy was performed using an inverted Olympus Fluoview 1000 laser scanning microscope. Final images were prepared using Image J v1.47m software (NIH, Bethesda, Md.).

Vaccine Efficacy Study

The efficacy of the experimental vaccine constructs was evaluated in K18-hACE2 mice (6 weeks of age) obtained from The Jackson Laboratory and maintained in bio-safety level-2 (BSL) containment pre-challenge and BSL-3 post challenge. At every indicated time point, mice were concurrently immunized with MVA or pQAC S and N constructs. In the primary trial, a total of 60 mice was divided equally into four groups (n=15 each). Groups of mice were either unvaccinated (PBS) or immunized with pQAC-CoV (IM) or pQAC-CoV (IN) at week-0, and week-6. Another group of K18-hACE2 mice was vaccinated with pQAC-CoV (IN) at week-0, followed by a boost with MVA-CoV (IN) at week-6. A vaccine dose of 50 ugs/plasmid DNA construct/animal, and 108 pfu/MVA construct/animal was administered at each immunization timepoint. Sera for neutralizing antibody titers were harvested from blood collected at week-9. At week-9, three weeks post final boost, pre-challenge, mice (n=4) were euthanized, BAL collected as described previously in D10 media, and lungs were harvested and processed for ICS assay as described below. At week-9, mice were challenged with SARS-CoV-2 isolate USA-WA1/2020 intranasally at a dosage of 104 PFU. Mice were weighed on the day of SARS-CoV-2 challenge and everyday thereafter. 5-6 mice were euthanized at 4- and 6-days post challenge (dpc). Mice were euthanized via isoflurane overdose and then cervical dislocation. Lung, spleen, and brain were collected for viral load quantitation and histopathology. A second follow-up study was conducted to validate the findings of the initial trial. In the follow-up trial, a total of 24 mice was divided equally into two groups (n=12 each). Groups of mice were either unvaccinated (PBS) or immunized with pQAC-CoV (IM) at week-0, and week-6 and challenged as described above at week-9. Samples were collected for vaccine efficacy read outs as detailed above.

SARS-CoV-2 Neutralization Assay

SARS-CoV-2, isolate USA-WA1/2020 (lineage A), or isolate SA/2020 (lineage B.1.351) or isolate England/2020 (lineage B1.1.7), kind gifts from Dr. Jorge Osorio was propagated and titrated on Vero E6 cells. Heat-inactivated sera and BAL were first serially diluted in serum-free Opti-MEM media and incubated with 100 PFU per well of SARS-CoV-2 isolates for 60 mins at 37° C. and transferred into wells pre-seeded with Vero E6 cells. Plates were incubated at 37° C. for four days before scoring for the cytopathic effect. Neutralization titer was calculated as the reciprocal of the highest dilution at which virus neutralization occurred.

Flow Cytometric Assessment of SARS-CoV-2 Specific Intracellular Cytokine Assay

Immunized K18-hACE2 mice (N=4) from each vaccine group 3 weeks post final boost was euthanized and used for flow cytometric assessment. Single-cell suspensions from the lungs were prepared using standard techniques were used. Briefly, lungs were excised and placed in a gentle MACS dissociator M tube (Miltenyi 130-093-236) with 3 mL collagenase B (1 mg/ml, Roche). Lung tissue was processed using the gentle MACS dissociator, followed by incubation for 30 min at 37° C. Single-cell suspensions lung were prepared by gently squeezing through a 70-mm cell strainer (Falcon) after lysing RBCs using 1×BD Biosciences BD Pharm Lyse™. For intracellular cytokine staining, 1×106 cells were stimulated with SARS-CoV-2 Spike protein (BEIre-sources-NR-52396, 100 ng total/well) or N Protein N-terminal RNA binding domain (BEIresources-NR-53246, 100 ng total/well) overnight (˜18 hrs) at 37° C. Brefeldin A (1 μl/ml, Golgi Plug, BD Biosciences) was added after, and cells were further incubated for another 5 hours at 37° C. Fluorochrome-labeled antibodies against the cell-surface antigens CD4 (BUV 496, GK1.5), CD8a (BUV395, 53-6.7) and intracellular antigens IFN-γ (APC, XMG1.2), TNF-α (BV421, MP6-XT22), IL-2 (PE-CF594, JES6-5H4), IL-17 (FITC, TC11-18H10.1), IL-13 (PE-Cy7, eBio13A) or IL-4 (PerCP-Cy5.5, 11B11) were purchased from BD Biosciences (San Jose, Calif.), Biolegend (San Diego, Calif.), eBioscience (San Diego, Calif.) or Invitrogen (Grand Island, N.Y.). Before antibody staining, cells were stained for viability with Dye eFluor 780 (eBiosciences, San Diego, Calif.). After stimulation, cells were stained for surface markers and then processed with the Cytofix/Cytoperm kit (BD Biosciences, NJ). To stain for cytokines, cells were first stained for cell surface molecules, fixed, permeabilized, and subsequently stained for the cytokines. All samples were acquired on an LSR Fortessa (BD Biosciences) flow cytometer. Data were analyzed with FlowJo software (TreeStar, OR). Results are expressed as the difference in the percentage of stimulated cells with that of unstimulated cells. At least 100,000 events were collected for each sample. A boolean gating strategy was applied for the determination of cytokine secreting T cells.

Viral Load Measurement

Tissue samples were homogenized to a final 1 mL suspension in serum free media (Opti-MEM) with sterile zirconia beads, clarified by low speed centrifugation at 800×g for 10 min at 4° C., and virus titers were determined in Vero E6 cell monolayers grown in 96-well plates. Vero E6 cells were seeded (0.25×105/well) in a 96-well plate and incubated overnight at 37° C. in a CO2 incubator. 1004, of 10-fold serially diluted tissue suspension was added to each well in quadruplicate format for 1 hr at 37° C. and replaced with fresh complete DMEM media. Plates were incubated in a CO2 incubator at 37° C. for 3-4 days, after which cytopathic effect (CPE) was observed microscopically at 40× magnification. Virus titers were expressed as TCID50 units per gram of tissue and then converted to PFU/ml by multiplying the TCID50/mL by 0.7 ( ). For qRT-PCR, RNA was extracted from homogenized brain samples (see above, 100 μl) using ZymoDirect-Zol™ RNA mini prep kit (Zymo Research, CA, USA) according to manufacturer's instructions. RT-qPCR was conducted in two steps: cDNA synthesis (Invitrogen™ SuperScript™ III First-Strand Synthesis System) and qPCR reactions. cDNA synthesis was performed with 0.5 μl (50 ng/μl) random hexamers, 0.5 μl of 10 mM dNTPs, and 4 μl RNA and heated at 65° C. for 5 min and chilled on ice followed by addition of 1 μl of 10×RT buffer, 1 μl of 0.1 M DTT, 1 μla 25 mM MgCl2, 0.5 μl of RNaseOUT and 0.5 μl of SuperScript III enzyme in final volume of 10 μl. The reaction conditions include 25° C. for 5 min, 50° C. for 60 min and 70° C. for 15 min. SYBR green RT-qPCR was performed using SARS-CoV-2 N gene specific primer pair set forward primer: 5′ GACCCCAAA ATCAGCGAAAT 3′ (SEQ ID NO: 7) and reverse primer: 5′TCTGGTTACTGCCAGTTGAATCTG 3′ (SEQ ID NO: 8). PCRs were performed using a StepOnePlus™ Real-Time PCR System (Applied Biosystems, Foster City, Calif., U.S.A) under the following conditions: one cycle 95 C for 2 min followed by 40 cycles of 95 C for 3 sec and 60 C for 30 sec. Each 20 μl reaction was carried out using 1 μl of diluted cDNA (1/10), 10 μl of GoTaq® qPCR mastermix (Promega), 2 μl of forward and reverse primers and 7 μl of nuclease free water. A serial 10-fold dilution of cDNA extracted from quantitative PCR (qPCR) Control RNA from Heat-Inactivated SARS-CoV-2, Isolate USA-WA1/2020 (BEI resources, NR-52347) was used to establish the standard curve. Temperature melt curve analysis was used to confirm the specificity of the product.

Histopathological Analysis

Lungs, spleen, and brain were collected from the different experimental groups were routinely processed. The paraffin embedded blocks were sectioned at 5-micron thickness and stained with Hematoxylin and Eosin (Bancroft et al. 2010) for histopathological examination by alight microscope. For Lung histopathological analysis, perivascular lymphoid aggregates were ordinally scored: 0, absent; 1, few solitary cells; 2, moderate small to medium aggregates; or 3, robust aggregates forming circumferential perivascular cuffs with compression of adjacent parenchyma. Interstitial disease was ordinally scored using a modified H score: 0, absent; 1, minor scattered cells in septa; 2, moderate infiltrates in septa and extending into lumen; or 3, moderate to severe infiltrates in septa and lumen with associated consolidation/atelectasis and or edema. For each of the tiers, the percentage of lung affected was recorded ( ). For spleen scoring, histopathological alterations in spleen were scored as, no changes (0), mild (1), moderate (2) and severe (3) changes, while the grading was determined by percentage as follows: <30% changes were indicated as mild, <30%-50% indicated as moderate changes, and >50% indicated as severe changes (Arsad et al. 2014).

1. A vaccine composition comprising an adjuvant and a polynucleotide that encodes a SARs-CoV-2 spike (S) protein or portion thereof, a SARs-CoV-2 nucleocapsid (N) protein, or both the S protein and the N protein, wherein the adjuvant comprises disaggregated spherical nanostructures comprising Quil-A and chitosan, and wherein the Quil-A and chitosan are present at a ratio between 1:15 and 1:100.

2. The vaccine composition of claim 1, wherein the chitosan is functionalized by treatment with 5-formyl-2-furan sulfonic acid and sodium borohydride, such that the chitosan surface is negatively charged.

3. The vaccine composition of claim 1, wherein the spherical nanostructures are between about 5 nm and about 100 nm in diameter in the absence of a payload molecule.

4. The vaccine composition of claim 1, wherein the S protein comprises SEQ ID NO:2.

5. The vaccine composition of claim 1, wherein the S protein consists of SEQ ID NO:4.

6. The vaccine composition of claim 1, wherein the N protein comprises SEQ ID NO:6.

7. (canceled)

8. (canceled)

9. A vaccine composition comprising a viral vector, the viral vector comprising a polynucleotide encoding a SARs-CoV-2 spike (S) protein or a portion thereof, a SARs-CoV-2 nucleocapsid (N) protein, or both the S protein and the N protein.

10. The vaccine composition of claim 9, wherein the viral vector is selected from an adeno-associated virus or a poxvirus.

11. The vaccine composition of claim 10, wherein the viral vector is a modified vaccinia Ankara (MVA) virus.

12. The vaccine composition of claim 9, wherein the S protein comprises SEQ ID NO:2.

13. The vaccine composition of claim 9, wherein the S protein consists of SEQ ID NO:4.

14. The vaccine composition of claim 9, wherein the N protein comprises SEQ ID NO:6.

15. (canceled)

16. (canceled)

17. A method of inducing an immune response against SARs-CoV-2 in a subject, the method comprising: administering the vaccine composition of claim 1 in an amount effective to induce the immune response against at least one SARS-CoV-2 antigen in the subject.

18. The method of claim 17, wherein the administration is by an intranasal route or an intramuscular route.

19. (canceled)

20. A method of inducing an immune response against a SARs-CoV-2 in a subject, the method comprising:
a) administering a first vaccine composition comprising the vaccine composition of claim 1, and
b) administering a second vaccine composition comprising the vaccine composition of claim 9,
wherein administration of the first vaccine composition and the second vaccine composition induces the immune response against at least one SARS-CoV-2 antigen in the subject.

21. The method of claim 20, wherein administration of the second vaccine composition occurs at least three weeks after administration of the first vaccine composition.

22. (canceled)

23. (canceled)

24. (canceled)

25. The method of claim 20, wherein both the first vaccine composition and the second vaccine composition are administered by an intranasal route.

26. The method of claim 20, wherein both the first vaccine composition and the second vaccine composition are administered by an intramuscular route.

27. The method of claim 20, wherein the first vaccine composition is administered by an intranasal route and the second vaccine composition is administered by an intramuscular route.

28. The method of claim 20, wherein the first vaccine composition is administered by an intramuscular route and the second vaccine composition is administered by an intranasal route.

29. (canceled)

30. (canceled)

31. A method of inducing an immune response against SARs-CoV-2 in a subject, the method comprising: administering the vaccine composition of claim 9 in an amount effective to induce the immune response against at least one SARS-CoV-2 antigen in the subject.


SEQUENCE LISTING (8 sequences)

SEQ ID NO: 1 - DNA, 3849 bp, Artificial Sequence
  Synthetic- SARS-CoV-2 spike nucleotide sequence
atgtttgtgttcctggtactcctaccgctggtgagctcccaatgcgtcaatctcacgactaggacacaactcccacccgcctacacgaactcttttacaaggggtgtatactaccctgacaaggtgttccgcagcagcgtgctacatagcactcaagatttgtttctgcccttcttctctaacgtaacatggttccatgcgatacatgtatctggtaccaatggaacaaaacggtttgataacccagttctcccgtttaatgatggagtttactttgcatccactgaaaagtctaatataatacgcggatggattttcggcacaacactggatagtaagacccagagtctgcttattgtcaataacgccactaacgtggtgattaaggtgtgtgagtttcaattctgtaatgacccatttctcggggtttactaccataagaacaataagagctggatggaatcagaatttagagtttatagcagcgcaaacaattgcacatttgaatacgtttctcaaccctttctgatggatctcgaaggaaaacaagggaactttaagaacctcagggaatttgtcttcaagaacatagatgggtacttcaaaatttacagcaagcatacaccaattaacttggttagagacctgccccagggattttctgcacttgaacccttggttgacttgcctatcggcattaatattactcgatttcaaactctgctcgctttgcaccgcagctacctgacaccgggagactcctctagtggctggaccgcaggagcagctgcatattatgtgggttatctccaacccagaacatttctgctaaagtacaacgaaaatgggacaatcaccgatgcggtcgactgcgcattggatcccctatccgagactaagtgtactttgaagagctttaccgtggaaaaggggatctatcaaacttccaactttcgggtgcaaccgaccgaaagcatcgttagatttcccaatatcaccaacttgtgtccctttggcgaagttttcaacgctactcgctttgcaagtgtctatgcttggaatagaaagaggataagcaactgcgtcgctgactattctgttctgtataactctgcgagcttttcaactttcaaatgctatggggtctccccaaccaagcttaatgacttgtgcttcactaacgtttatgcagatagcttcgtcatacggggcgatgaagtgcggcaaatcgcgccaggacaaacaggtaagatcgccgactacaactataaactgccagacgattttacaggctgcgtgatcgcttggaactctaacaatcttgattctaaagttggtgggaattataactatctatatcgtctcttcagaaaatctaacctcaagccctttgagcgggatataagcacagaaatctatcaggctgggtctactccttgcaatggtgtcgaaggtttcaattgctactttccactgcaatcctacggctttcaacctacaaatggtgtcggttatcaaccgtatagagttgttgtgctctcctttgagttgctacacgctcctgccacggtttgtggacccaagaagtcaacgaatctggtgaagaataaatgcgtcaattttaattttaacggacttacggggactggcgttctcaccgaatccaataagaagttccttccattccagcaatttggccgcgatatcgccgatacaacagacgctgtcagagatccacaaaccttggagatcttggatattacaccatgctctttcggcggggtatcagtaatcacgccggggacaaatacatccaatcaggttgccgtattgtatcaggatgtgaactgcaccgaggtccctgttgccatacatgccgatcagttgactccgacttggcgggtgtattctacagggtctaatgtatttcaaaccagagcaggatgcctcatcggtgccgaacatgttaataattcatatgagtgtgatatccccataggagcaggcatttgtgcatcctaccaaactcagaccaactcaccaagacgggcccggtctgtggcgtcccagtccataattgcctacacaatgagtcttggagccgaaaactcagttgcctattccaacaactccatcgctatccccacgaattttacaatctcagttaccactgaaatcctgcccgtatctatgacaaagacaagtgttgattgcacaatgtatatctgcggggactctactgagtgctcaaatctgctgctacagtacggtagcttttgtactcagctaaatcgcgcactcaccgggattgctgtagaacaagacaagaatacacaagaggttttcgcccaagtgaaacaaatatacaaaacgccccctataaaagacttcggtggttttaactttagccagattttgccggatccatccaaaccatctaagcgctctttcattgaagatctcctgttcaacaaagttacattggcggacgcaggtttcattaagcaatatggggattgccttggggatatcgcagcgcgggatctgatttgtgcccagaagttcaatggtctgacggttctgccacccctactcaccgatgaaatgatcgcgcaatataccagcgctcttctggcaggtaccatcacttccggctggaccttcggagccggcgccgcacttcaaatccctttcgccatgcaaatggcatatagatttaatggcatcggtgtcacccaaaacgtattgtatgagaaccaaaagctgattgccaatcaatttaatagcgcaattggtaaaatacaagacagcctgagcagtacggcaagcgcactcgggaagcttcaagatgtagttaatcagaatgctcaggctttgaatactcttgtgaaacaactctcttccaattttggcgccatctcatctgtgctcaacgacattctttcccgactcgataaggtggaagctgaagttcaaatcgatcggctcattacaggcagactgcagtctctccaaacctacgtcacccagcaactgattagggcggcagaaattcgcgcttctgcaaatcttgcagccacaaagatgagcgagtgtgtcctgggccaatcaaagcgcgtcgacttttgcggaaaggggtatcatctcatgagctttccacaatcagccccacatggagttgtctttctccatgtaacgtacgtccctgctcaggaaaagaatttcaccacagccccagctatttgccatgacggaaaggctcacttcccacgtgaaggcgtatttgtatcaaatggcacccactggttcgtcacccaacgtaacttttacgagcctcagattatcaccacggataacacgttcgtcagcgggaattgtgatgtagtcattggtattgtcaataataccgtgtatgatccccttcaacccgaattggactcctttaaagaagaactcgacaaatactttaagaaccatacgtcccctgacgtggacctcggtgacatttcaggcataaatgcctcagtggtgaacattcaaaaggaaatcgataggctgaacgaagtggccaagaatttgaacgaaagtttgattgatctccaagaactggggaaatacgaacaatatataaaatggccttggtatatctggcttggatttatagcagggcttattgctattgtaatggtcactatcatgctctgttgtatgacctcttgttgctcctgcctaaaaggatgttgctcttgtgggtcctgctgcaaatttgacgaggatgacagtgagccagtactgaaaggggtcaagctccattacacaggtggcggacaccatcatcatcaccattaa

SEQ ID NO: 2 - protein, 1282 aa, Artificial Sequence
  Synthetic- SARS-CoV-2 spike amino acid sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTGGGHHHHHH

SEQ ID NO: 3 - DNA, 3666 bp, Artificial Sequence
  Synthetic- SARS-CoV-2 truncated spike nucleotide sequence
atgtttgtgttcctggtactcctaccgctggtgagctcccaatgcgtcaatctcacgactaggacacaactcccacccgcctacacgaactcttttacaaggggtgtatactaccctgacaaggtgttccgcagcagcgtgctacatagcactcaagatttgtttctgcccttcttctctaacgtaacatggttccatgcgatacatgtatctggtaccaatggaacaaaacggtttgataacccagttctcccgtttaatgatggagtttactttgcatccactgaaaagtctaatataatacgcggatggattttcggcacaacactggatagtaagacccagagtctgcttattgtcaataacgccactaacgtggtgattaaggtgtgtgagtttcaattctgtaatgacccatttctcggggtttactaccataagaacaataagagctggatggaatcagaatttagagtttatagcagcgcaaacaattgcacatttgaatacgtttctcaaccctttctgatggatctcgaaggaaaacaagggaactttaagaacctcagggaatttgtcttcaagaacatagatgggtacttcaaaatttacagcaagcatacaccaattaacttggttagagacctgccccagggattttctgcacttgaacccttggttgacttgcctatcggcattaatattactcgatttcaaactctgctcgctttgcaccgcagctacctgacaccgggagactcctctagtggctggaccgcaggagcagctgcatattatgtgggttatctccaacccagaacatttctgctaaagtacaacgaaaatgggacaatcaccgatgcggtcgactgcgcattggatcccctatccgagactaagtgtactttgaagagctttaccgtggaaaaggggatctatcaaacttccaactttcgggtgcaaccgaccgaaagcatcgttagatttcccaatatcaccaacttgtgtccctttggcgaagttttcaacgctactcgctttgcaagtgtctatgcttggaatagaaagaggataagcaactgcgtcgctgactattctgttctgtataactctgcgagcttttcaactttcaaatgctatggggtctccccaaccaagcttaatgacttgtgcttcactaacgtttatgcagatagcttcgtcatacggggcgatgaagtgcggcaaatcgcgccaggacaaacaggtaagatcgccgactacaactataaactgccagacgattttacaggctgcgtgatcgcttggaactctaacaatcttgattctaaagttggtgggaattataactatctatatcgtctcttcagaaaatctaacctcaagccctttgagcgggatataagcacagaaatctatcaggctgggtctactccttgcaatggtgtcgaaggtttcaattgctactttccactgcaatcctacggctttcaacctacaaatggtgtcggttatcaaccgtatagagttgttgtgctctcctttgagttgctacacgctcctgccacggtttgtggacccaagaagtcaacgaatctggtgaagaataaatgcgtcaattttaattttaacggacttacggggactggcgttctcaccgaatccaataagaagttccttccattccagcaatttggccgcgatatcgccgatacaacagacgctgtcagagatccacaaaccttggagatcttggatattacaccatgctctttcggcggggtatcagtaatcacgccggggacaaatacatccaatcaggttgccgtattgtatcaggatgtgaactgcaccgaggtccctgttgccatacatgccgatcagttgactccgacttggcgggtgtattctacagggtctaatgtatttcaaaccagagcaggatgcctcatcggtgccgaacatgttaataattcatatgagtgtgatatccccataggagcaggcatttgtgcatcctaccaaactcagaccaactcaccaagacgggcccggtctgtggcgtcccagtccataattgcctacacaatgagtcttggagccgaaaactcagttgcctattccaacaactccatcgctatccccacgaattttacaatctcagttaccactgaaatcctgcccgtatctatgacaaagacaagtgttgattgcacaatgtatatctgcggggactctactgagtgctcaaatctgctgctacagtacggtagcttttgtactcagctaaatcgcgcactcaccgggattgctgtagaacaagacaagaatacacaagaggttttcgcccaagtgaaacaaatatacaaaacgccccctataaaagacttcggtggttttaactttagccagattttgccggatccatccaaaccatctaagcgctctttcattgaagatctcctgttcaacaaagttacattggcggacgcaggtttcattaagcaatatggggattgccttggggatatcgcagcgcgggatctgatttgtgcccagaagttcaatggtctgacggttctgccacccctactcaccgatgaaatgatcgcgcaatataccagcgctcttctggcaggtaccatcacttccggctggaccttcggagccggcgccgcacttcaaatccctttcgccatgcaaatggcatatagatttaatggcatcggtgtcacccaaaacgtattgtatgagaaccaaaagctgattgccaatcaatttaatagcgcaattggtaaaatacaagacagcctgagcagtacggcaagcgcactcgggaagcttcaagatgtagttaatcagaatgctcaggctttgaatactcttgtgaaacaactctcttccaattttggcgccatctcatctgtgctcaacgacattctttcccgactcgataaggtggaagctgaagttcaaatcgatcggctcattacaggcagactgcagtctctccaaacctacgtcacccagcaactgattagggcggcagaaattcgcgcttctgcaaatcttgcagccacaaagatgagcgagtgtgtcctgggccaatcaaagcgcgtcgacttttgcggaaaggggtatcatctcatgagctttccacaatcagccccacatggagttgtctttctccatgtaacgtacgtccctgctcaggaaaagaatttcaccacagccccagctatttgccatgacggaaaggctcacttcccacgtgaaggcgtatttgtatcaaatggcacccactggttcgtcacccaacgtaacttttacgagcctcagattatcaccacggataacacgttcgtcagcgggaattgtgatgtagtcattggtattgtcaataataccgtgtatgatccccttcaacccgaattggactcctttaaagaagaactcgacaaatactttaagaaccatacgtcccctgacgtggacctcggtgacatttcaggcataaatgcctcagtggtgaacattcaaaaggaaatcgataggctgaacgaagtggccaagaatttgaacgaaagtttgattgatctccaagaactggggaaatacgaacaatatataaaatggggtggcggacaccatcatcatcaccattaa

SEQ ID NO: 4 - protein, 1221 aa, Artificial Sequence
  Synthetic- SARS-CoV-2 truncated spike amino acid sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWGGGHHHHHH

SEQ ID NO: 5 - DNA, 1287 bp, Artificial Sequence
  Synthetic- SARS-CoV-2 nucleocapsid nucleotide sequence
atgagcgataacggaccccagaaccaacgtaacgcccctcgcattacttttggcgggccttccgactctacaggttctaatcaaaacggggagcgaagcggggcacgcagcaagcaacgcaggcctcaaggtctgccaaacaacactgcttcttggttcactgctctcactcaacatggcaaagaggatctgaaattcccccgaggacaaggggtaccgatcaatactaactcctctcccgatgatcaaattggatactacaggcgcgccacccggagaattcgcggcggagacgggaaaatgaaggaccttagtccaagatggtacttttattacttggggaccggaccggaagctggcttgccctatggtgccaataaggacggaattatttgggtggcgacagagggcgctctgaatactcccaaggaccacataggtactcggaatcctgctaataatgccgccatcgtgttgcagctcccccaaggcaccaccctacccaagggcttttatgccgaggggtcccgcggcgggagccaggcttcatccaggagttctagtcgctctcgcaactcatcccgcaactccacccctggctctagtaggggtactagtcccgcccgaatggccggcaatggcggagacgctgccctagcgctgttgttgctggatagactcaatcagctcgaaagtaagatgtccggcaaaggacaacagcagcaaggacaaacggttaccaagaaaagcgcagcagaggcaagtaagaaacccaggcaaaagcgcactgccaccaaagcttacaacgtgacacaggcctttggtcgcagaggacccgaacaaacacaaggcaattttggagatcaggaacttataaggcaaggaacagactataagcattggccccaaattgcacaatttgccccatccgcttctgcattcttcggtatgagtaggattggaatggaagttacacccagtggcacctggctcacatatacaggcgctatcaagcttgatgataaggatccgaattttaaggaccaggtgatcctgttgaacaaacatatagacgcttataaaacctttcccccgactgaacctaagaaagacaagaagaagaaagctgatgaaactcaagcgctgccgcaaagacaaaagaaacaacaaaccgttacgctcctgccagcagctgacctggacgattttagtaaacaactccaacaaagcatgtctagcgccgatagtactcaggctggcggcgggcaccaccatcatcatcactga

SEQ ID NO: 6 - protein, 428 aa, Artificial Sequence
  Synthetic- SARS-CoV-2 nucleocapsid amino acid sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGHHHHHH

SEQ ID NO: 7 - DNA, 20 bp, Artificial Sequence
  Synthetic- SARS-CoV-2 N F
gaccccaaaatcagcgaaat

SEQ ID NO: 8 - DNA, 24 bp, Artificial Sequence
  Synthetic- SARS-CoV-2 N R
tctggttactgccagttgaatctg